SEQUENCE LISTING (180 sequences)

SEQ ID NO: 1 - protein, 199 aa, Streptomyces alboniger
TEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAN

SEQ ID NO: 2 - DNA, 597 bp, Streptomyces alboniger
  source 1..597: mol_type = genomic DNA
accgagtacaagcccacggtgcgcctcgccacccgcgacgacgtcccgcgggccgtacgcaccctcgccgccgcgttcgccgactaccccgccacgcgccacaccgtagacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccaac

SEQ ID NO: 3 - protein, 7 aa, synthetic construct
  REGION 1..7: note = spacer sequence
SGRSGGG

SEQ ID NO: 4-5 - skipped (2 entries)

SEQ ID NO: 6 - protein, 5 aa, synthetic construct
  REGION 1..5: note = GSGGS linker
  REGION >5: note = The entire sequence of amino acids 1-5 can be repeated one or more times.
GSGGS

SEQ ID NO: 7 - protein, 4 aa, synthetic construct
  REGION 1..4: note = GGGS linker
  REGION >4: note = The entire sequence of amino acids 1-4 can be repeated one or more times.
GGGS

SEQ ID NO: 8 - protein, 4 aa, synthetic construct
  REGION 1..4: note = GGSG linker
GGSG

SEQ ID NO: 9 - protein, 5 aa, synthetic construct
  REGION 1..5: note = GGSGG linker
GGSGG

SEQ ID NO: 10 - protein, 5 aa, synthetic construct
  REGION 1..5: note = GSGSG linker
GSGSG

SEQ ID NO: 11 - protein, 5 aa, synthetic construct
  REGION 1..5: note = GSGGG linker
GSGGG

SEQ ID NO: 12 - protein, 5 aa, synthetic construct
  REGION 1..5: note = GGGSG linker
GGGSG

SEQ ID NO: 13 - protein, 5 aa, synthetic construct
  REGION 1..5: note = GSSSG linker
GSSSG

SEQ ID NO: 14 - protein, 18 aa, Thosea Asigna Virus
EGRGSLLTCGDVEENPGP

SEQ ID NO: 15 - DNA, 54 bp, Thosea Asigna Virus
  source 1..54: mol_type = other DNA
gagggcagaggaagtctgctaacatgcggtgacgtcgaggagaatcctggccca

SEQ ID NO: 16 - protein, 20 aa, Thosea Asigna Virus
RAEGRGSLLTCGDVEENPGP

SEQ ID NO: 17 - DNA, 60 bp, Thosea Asigna Virus
  source 1..60: mol_type = other DNA
agggccgagggcagaggaagtctgctaacatgcggtgacgtcgaggagaatcctggccca

SEQ ID NO: 18 - protein, 20 aa, Thosea Asigna Virus
SGEGRGSLLTCGDVEENPGP

SEQ ID NO: 19 - DNA, 60 bp, Thosea asigna virus
  source 1..60: mol_type = other DNA
tccggcgagggcagaggaagtctgctaacatgcggtgacgtcgaggagaatcctggccca

SEQ ID NO: 20 - protein, 24 aa, Thosea Asigna Virus
LEGGGEGRGSLLTCGDVEENPGPR

SEQ ID NO: 21 - DNA, 72 bp, Thosea Asigna Virus
  source 1..72: mol_type = other DNA
ctggagggcggcggcgagggcagaggaagtctgctaacatgcggtgacgtcgaggagaatcctggcccaagg

SEQ ID NO: 22 - protein, 14 aa, porcine teschovirus-1
LLKQAGDVEENPGP

SEQ ID NO: 23 - DNA, 42 bp, porcine teschovirus-1
  source 1..42: mol_type = other DNA
ctgctgaagcaggccggcgacgtggaggagaaccccggcccc

SEQ ID NO: 24 - protein, 19 aa, porcine teschovirus-1
ATNFSLLKQAGDVEENPGP

SEQ ID NO: 25 - DNA, 57 bp, porcine teschovirus-1
  source 1..57: mol_type = other DNA
gccaccaacttctccctgctgaagcaggccggcgacgtggaggagaaccccggcccc

SEQ ID NO: 26 - protein, 21 aa, porcine teschovirus-1
SGATNFSLLKQAGDVEENPGP

SEQ ID NO: 27 - DNA, 63 bp, porcine teschovirus-1
  source 1..63: mol_type = other DNA
tccggcgccaccaacttctccctgctgaagcaggccggcgacgtggaggagaaccccggcccc

SEQ ID NO: 28 - protein, 14 aa, equine rhinitis A virus
LLKLAGDVESNPGP

SEQ ID NO: 29 - DNA, 42 bp, Equine rhinitis A virus
  source 1..42: mol_type = other DNA
ctgctgaagctggccggcgacgtggagtccaaccccggcccc

SEQ ID NO: 30 - protein, 20 aa, equine rhinitis A virus
QCTNYALLKLAGDVESNPGP

SEQ ID NO: 31 - DNA, 60 bp, equine rhinitis A virus
  source 1..60: mol_type = other DNA
cagtgcaccaactacgccctgctgaagctggccggcgacgtggagtccaaccccggcccc

SEQ ID NO: 32 - protein, 22 aa, equine rhinitis A virus
SGQCTNYALLKLAGDVESNPGP

SEQ ID NO: 33 - DNA, 66 bp, equine rhinitis A virus
  source 1..66: mol_type = other DNA
tccggccagtgcaccaactacgccctgctgaagctggccggcgacgtggagtccaaccccggcccc

SEQ ID NO: 34 - protein, 17 aa, Foot-and-mouth disease virus
NFDLLKLAGDVESNPGP

SEQ ID NO: 35 - DNA, 51 bp, Foot-and-mouth disease virus
  source 1..51: mol_type = other DNA
aacttcgacctgctgaagctggccggcgacgtggagtccaaccccggcccc

SEQ ID NO: 36 - protein, 18 aa, Foot-and-mouth disease virus
LNFDLLKLAGDVESNPGP

SEQ ID NO: 37 - DNA, 54 bp, Foot-and-mouth disease virus
  source 1..54: mol_type = other DNA
ctgaacttcgacctgctgaagctggccggcgacgtggagtccaaccccggcccc

SEQ ID NO: 38 - protein, 19 aa, Foot-and-mouth disease virus
LLNFDLLKLAGDVESNPGP

SEQ ID NO: 39 - DNA, 57 bp, Foot-and-mouth disease virus
  source 1..57: mol_type = other DNA
ctgctgaacttcgacctgctgaagctggccggcgacgtggagtccaaccccggcccc

SEQ ID NO: 40 - protein, 19 aa, Foot-and-mouth disease virus
TLNFDLLKLAGDVESNPGP

SEQ ID NO: 41 - DNA, 57 bp, Foot-and-mouth disease virus
  source 1..57: mol_type = other DNA
accctgaacttcgacctgctgaagctggccggcgacgtggagtccaaccccggcccc

SEQ ID NO: 42 - protein, 20 aa, Foot-and-mouth disease virus
QLLNFDLLKLAGDVESNPGP

SEQ ID NO: 43 - DNA, 60 bp, Foot-and-mouth disease virus
  source 1..60: mol_type = other DNA
cagctgctgaacttcgacctgctgaagctggccggcgacgtggagtccaaccccggcccc

SEQ ID NO: 44 - protein, 22 aa, Foot-and-mouth disease virus
VKQTLNFDLLKLAGDVESNPGP

SEQ ID NO: 45 - DNA, 66 bp, Foot-and-mouth disease virus
  source 1..66: mol_type = other DNA
gtgaaacagactttgaattttgaccttctcaagttggcgggagacgtggagtccaacccagggccg

SEQ ID NO: 46 - protein, 24 aa, Foot-and-mouth disease virus
APVKQTLNFDLLKLAGDVESNPGP

SEQ ID NO: 47 - DNA, 72 bp, Foot-and-mouth disease virus
  source 1..72: mol_type = other DNA
gcccccgtgaagcagaccctgaacttcgacctgctgaagctggccggcgacgtggagtccaaccccggcccc

SEQ ID NO: 48 - protein, 24 aa, Foot-and-mouth disease virus
SGVKQTLNFDLLKLAGDVESNPGP

SEQ ID NO: 49 - DNA, 72 bp, Foot-and-mouth disease virus
  source 1..72: mol_type = other DNA
tccggcgtgaagcagaccctgaacttcgacctgctgaagctggccggcgacgtggagtccaaccccggcccc

SEQ ID NO: 50 - protein, 33 aa, Foot-and-mouth disease virus
EARHKQKIVAPVKQTLNFDLLKLAGDVESNPGP

SEQ ID NO: 51 - DNA, 99 bp, Foot-and-mouth disease virus
  source 1..99: mol_type = other DNA
gaggccaggcacaagcagaagatcgtggcccccgtgaagcagaccctgaacttcgacctgctgaagctggccggcgacgtggagtccaaccccggcccc

SEQ ID NO: 52 - protein, 40 aa, Foot-and-mouth disease virus
LLAIHPTEARHKQKIVAPVKQTLNFDLLKLAGDVESNPGP

SEQ ID NO: 53 - DNA, 120 bp, Foot-and-mouth disease virus
  source 1..120: mol_type = other DNA
ctgctggccatccaccccaccgaggccaggcacaagcagaagatcgtggcccccgtgaagcagaccctgaacttcgacctgctgaagctggccggcgacgtggagtccaaccccggcccc

SEQ ID NO: 54 - protein, 20 aa, Acute bee paralysis virus
GSWTDILLLLSGDVETNPGP

SEQ ID NO: 55 - DNA, 60 bp, Acute bee paralysis virus
  source 1..60: mol_type = other DNA
ggctcctggaccgacatcctgctgctgctgtccggcgacgtggagaccaaccccggcccc

SEQ ID NO: 56 - protein, 30 aa, Acute bee paralysis virus
TGFLNKLYHCGSWTDILLLLSGDVETNPGP

SEQ ID NO: 57 - DNA, 90 bp, Acute bee paralysis virus
  source 1..90: mol_type = other DNA
accggcttcctgaacaagctgtaccactgcggctcctggaccgacatcctgctgctgctgtccggcgacgtggagaccaaccccggcccc

SEQ ID NO: 58 - protein, 19 aa, Drosophila C virus
AARQMLLLLSGDVETNPGP

SEQ ID NO: 59 - DNA, 57 bp, Drosophila C virus
  source 1..57: mol_type = other DNA
gccgccaggcagatgctgctgctgctgtccggcgacgtggagaccaaccccggcccc

SEQ ID NO: 60 - protein, 30 aa, infectious myonecrosis virus
WDPTYIEISDCMLPPPDLTSCGDVESNPGP

SEQ ID NO: 61 - DNA, 90 bp, infectious myonecrosis virus
  source 1..90: mol_type = other DNA
tgggaccccacctacatcgagatctccgactgcatgctgcccccccccgacctgacctcctgcggcgacgtggagtccaaccccggcccc

SEQ ID NO: 62 - protein, 30 aa, infectious myonecrosis virus
RDVRYIEKPFDKEEHTDILLSGDVEENPGP

SEQ ID NO: 63 - DNA, 90 bp, infectious myonecrosis virus
  source 1..90: mol_type = other DNA
agggacgtgaggtacatcgagaagcccttcgacaaggaggagcacaccgacatcctgctgtccggcgacgtggaggagaaccccggcccc

SEQ ID NO: 64 - protein, 20 aa, synthetic construct
  REGION 1..20: note = artificial self-cleaving peptide sequence
SSIIRTKMLVSGDVEENPGP

SEQ ID NO: 65 - DNA, 60 bp, synthetic construct
  misc_feature 1..60: note = artificial self-cleaving peptide sequence
  source 1..60: mol_type = other DNA
tcctccatcatcaggaccaagatgctggtgtccggcgacgtggaggagaaccccggcccc

SEQ ID NO: 66 - protein, 20 aa, synthetic construct
  REGION 1..20: note = artificial self-cleaving peptide sequence
AKFQIDKILISGDVELNPGP

SEQ ID NO: 67 - DNA, 60 bp, synthetic construct
  misc_feature 1..60: note = artificial self-cleaving peptide sequence
  source 1..60: mol_type = other DNA
gccaagttccagatcgacaagatcctgatctccggcgacgtggagctgaaccccggcccc

SEQ ID NO: 68 - protein, 20 aa, synthetic construct
  REGION 1..20: note = artificial self-cleaving peptide sequence
GATNFSLLKLAGDVELNPGP

SEQ ID NO: 69 - DNA, 60 bp, synthetic construct
  misc_feature 1..60: note = artificial self-cleaving peptide sequence
  source 1..60: mol_type = other DNA
ggcgccaccaacttctccctgctgaagctggccggcgacgtggagctgaaccccggcccc

SEQ ID NO: 70 - protein, 20 aa, synthetic construct
  REGION 1..20: note = artificial self-cleaving peptide sequence
SKFQIDKILISGDIELNPGP

SEQ ID NO: 71 - DNA, 60 bp, synthetic construct
  misc_feature 1..60: note = artificial self-cleaving peptide sequence
  source 1..60: mol_type = other DNA
tccaagttccagatcgacaagatcctgatctccggcgacatcgagctgaaccccggcccc

SEQ ID NO: 72 - protein, 25 aa, synthetic construct
  REGION 1..25: note = artificial self-cleaving peptide sequence
KAVRGYHADYYKQRLIHDVEMNPGP

SEQ ID NO: 73 - DNA, 75 bp, synthetic construct
  misc_feature 1..75: note = artificial self-cleaving peptide sequence
  source 1..75: mol_type = other DNA
aaggccgtgaggggctaccacgccgactactacaagcagaggctgatccacgacgtggagatgaaccccggcccc

SEQ ID NO: 74 - protein, 20 aa, synthetic construct
  REGION 1..20: note = artificial self-cleaving peptide sequence
CDAQRQKLLLSGDIEQNPGP

SEQ ID NO: 75 - DNA, 60 bp, synthetic construct
  misc_feature 1..60: note = artificial self-cleaving peptide sequence
  source 1..60: mol_type = other DNA
tgcgacgcccagaggcagaagctgctgctgtccggcgacatcgagcagaaccccggcccc

SEQ ID NO: 76 - protein, 20 aa, synthetic construct
  REGION 1..20: note = artificial self-cleaving peptide sequence
FLRKRTQLLMSGDVESNPGP

SEQ ID NO: 77 - DNA, 60 bp, synthetic construct
  misc_feature 1..60: note = artificial self-cleaving peptide sequence
  source 1..60: mol_type = other DNA
ttcctgaggaagaggacccagctgctgatgtccggcgacgtggagtccaaccccggcccc

SEQ ID NO: 78 - protein, 20 aa, synthetic construct
  REGION 1..20: note = artificial self-cleaving peptide sequence
TRAEIEDELIRAGIESNPGP

SEQ ID NO: 79 - DNA, 60 bp, synthetic construct
  misc_feature 1..60: note = artificial self-cleaving peptide sequence
  source 1..60: mol_type = other DNA
accagggccgagatcgaggacgagctgatcagggccggcatcgagtccaaccccggcccc

SEQ ID NO: 80 - protein, 20 aa, synthetic construct
  REGION 1..20: note = artificial self-cleaving peptide sequence
HYAGYFADLLIHDIETNPGP

SEQ ID NO: 81 - DNA, 60 bp, synthetic construct
  misc_feature 1..60: note = artificial self-cleaving peptide sequence
  source 1..60: mol_type = other DNA
cactacgccggctacttcgccgacctgctgatccacgacatcgagaccaaccccggcccc

SEQ ID NO: 82 - protein, 25 aa, synthetic construct
  REGION 1..25: note = artificial self-cleaving peptide sequence
GIFNAHYAGYFADLLIHDIETNPGP

SEQ ID NO: 83 - DNA, 75 bp, synthetic construct
  misc_feature 1..75: note = artificial self-cleaving peptide sequence
  source 1..75: mol_type = other DNA
ggcatcttcaacgcccactacgccggctacttcgccgacctgctgatccacgacatcgagaccaaccccggcccc

SEQ ID NO: 84 - protein, 40 aa, synthetic construct
  REGION 1..40: note = artificial self-cleaving peptide sequence
VTELLYRMKRAETYCPRPLLAIHPTEARHKQKIVAPVKQT

SEQ ID NO: 85 - DNA, 120 bp, synthetic construct
  misc_feature 1..120: note = artificial self-cleaving peptide sequence
  source 1..120: mol_type = other DNA
gtgaccgagctgctgtacaggatgaagagggccgagacctactgccccaggcccctgctggccatccaccccaccgaggccaggcacaagcagaagatcgtggcccccgtgaagcagacc

SEQ ID NO: 86 - protein, 60 aa, Bos taurus
AQRPDFCLEPPYTGPCKARMIRYFYNAKAGLCQPFVYGGCRAKRNNFKSSEDCMRTCGGA

SEQ ID NO: 87 - DNA, 180 bp, Bos taurus
  source 1..180: mol_type = other DNA
gcccagcggcccgatttctgcctggaacctccatacaccggtccctgtaaagctaggatgatcagatatttttacaatgcaaaggccgggctgtgccaacctttcgtgtacggcggatgccgcgctaagcggaacaactttaaatcttccgaggactgtatgaggacttgcggtggcgcc

SEQ ID NO: 88 - protein, 6 aa, Homo sapiens
EEIIMD

SEQ ID NO: 89 - DNA, 18 bp, Homo sapiens
  source 1..18: mol_type = other DNA
gaggagatcatcatggac

SEQ ID NO: 90 - DNA, 21 bp, synthetic construct
  misc_feature 1..21: note = spacer coding nucleic acid
  source 1..21: mol_type = other DNA
tccggaagatctggcggcgga

SEQ ID NO: 91-93 - skipped (3 entries)

SEQ ID NO: 94 - protein, 4 aa, synthetic construct
  REGION 1..4: note = furin cleavage site
RQKR

SEQ ID NO: 95 - skipped

SEQ ID NO: 96 - DNA, 34 bp, synthetic construct
  misc_feature 1..34: note = L3
  source 1..34: mol_type = other DNA
ataacttcgtataaagtctcctatacgaagttat

SEQ ID NO: 97 - DNA, 34 bp, synthetic construct
  misc_feature 1..34: note = 2L
  source 1..34: mol_type = other DNA
ataacttcgtatagcatacattatacgaagttat

SEQ ID NO: 98 - DNA, 34 bp, synthetic construct
  misc_feature 1..34: note = loxFas
  source 1..34: mol_type = other DNA
ataacttcgtatagaaaggtatatacgaagttat

SEQ ID NO: 99 - protein, 5 aa, synthetic construct
  REGION 1..5: note = GGGGS linker
GGGGS

SEQ ID NO: 100 - DNA, 218 bp, Bos taurus
  source 1..218: mol_type = genomic DNA
ttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 101 - DNA, 75 bp, Homo sapiens
  source 1..75: mol_type = genomic DNA
ggcaggataatatatggtagggttcatagccagagtaacctttttttttaatttttattttattttatttttgag

SEQ ID NO: 102 - DNA, 418 bp, Simian virus 40
  source 1..418: mol_type = genomic DNA
gcagcaccatggcctgaaataacctctgaaagaggaacttggttaggttccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggactaggcttttgcaaaaagctag

SEQ ID NO: 103 - DNA, 608 bp, synthetic construct
  source 1..608: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 104 - DNA, 696 bp, synthetic construct
  source 1..696: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 105 - DNA, 2125 bp, Human cytomegalovirus
  source 1..2125: mol_type = genomic DNA
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 106 - protein, 32 aa, synthetic construct
  REGION 1..32: note = F-G4S2-T2A linker
RAKRGGGGSGGGGSEGRGSLLTCGDVEENPGP

SEQ ID NO: 107 - DNA, 96 bp, synthetic construct
  misc_feature 1..96: note = F-G4S2-T2A linker
  source 1..96: mol_type = other DNA
agagccaagcgcggcggcggcggcagcggcggcggcggcagcgagggcagaggaagtctgctaacatgcggtgacgtcgaggagaatcctggccca

SEQ ID NO: 108 - protein, 21 aa, synthetic construct
  REGION 1..21: note = GSG-T2A (1)
GSGEGRGSLLTCGDVEENPGP

SEQ ID NO: 109 - DNA, 63 bp, synthetic construct
  misc_feature 1..63: note = GSG-T2A (1)
  source 1..63: mol_type = other DNA
ggcagtggagagggcagaggaagtctgctaacatgcggtgacgtcgaggagaatcctggccca

SEQ ID NO: 110 - protein, 23 aa, synthetic construct
  REGION 1..23: note = GSG-E2A (2)
GSGQCTNYALLKLAGDVESNPGP

SEQ ID NO: 111 - DNA, 69 bp, synthetic construct
  misc_feature 1..69: note = GSG-E2A (2)
  source 1..69: mol_type = other DNA
ggcagtggacagtgcaccaactacgccctgctgaagctggccggcgacgtggagtccaaccccggcccc

SEQ ID NO: 112 - protein, 25 aa, synthetic construct
  REGION 1..25: note = GSG-F2A (6)
GSGVKQTLNFDLLKLAGDVESNPGP

SEQ ID NO: 113 - DNA, 75 bp, synthetic construct
  misc_feature 1..75: note = GSG-F2A (6)
  source 1..75: mol_type = other DNA
ggcagtggagtgaagcagaccctgaacttcgacctgctgaagctggccggcgacgtggagtccaaccccggcccc

SEQ ID NO: 114 - protein, 22 aa, synthetic construct
  REGION 1..22: note = GSG-P2A (2)
GSGATNFSLLKQAGDVEENPGP

SEQ ID NO: 115 - DNA, 66 bp, synthetic construct
  misc_feature 1..66: note = GSG-P2A (2)
  source 1..66: mol_type = other DNA
ggcagtggagccaccaacttctccctgctgaagcaggccggcgacgtggaggagaaccccggcccc

SEQ ID NO: 116 - protein, 4 aa, synthetic construct
  REGION 1..4: note = furin cleavage site RKRR
RKRR

SEQ ID NO: 117 - DNA, 12 bp, synthetic construct
  misc_feature 1..12: note = furin cleavage site RKRR
  source 1..12: mol_type = other DNA
cgcaaacggaga

SEQ ID NO: 118 - skipped

SEQ ID NO: 119 - protein, 4 aa, synthetic construct
  REGION 1..4: note = cleavable peptidic linker
LEAD

SEQ ID NO: 120 - protein, 4 aa, synthetic construct
  REGION 1..4: note = cleavable peptidic linker
LEPD

SEQ ID NO: 121 - protein, 4 aa, synthetic construct
  REGION 1..4: note = cleavable peptidic linker
LEAE

SEQ ID NO: 122 - protein, 5 aa, synthetic construct
  REGION 1..5: note = cleavable peptidic linker
IEPDI

SEQ ID NO: 123 - protein, 5 aa, synthetic construct
  REGION 1..5: note = cleavable peptidic linker
LEADT

SEQ ID NO: 124 - protein, 5 aa, synthetic construct
  REGION 1..5: note = cleavable peptidic linker
IEPDG

SEQ ID NO: 125 - protein, 5 aa, synthetic construct
  REGION 1..5: note = cleavable peptidic linker
IEPDV

SEQ ID NO: 126 - protein, 5 aa, synthetic construct
  REGION 1..5: note = cleavable peptidic linker
IEPDS

SEQ ID NO: 127 - protein, 5 aa, synthetic construct
  REGION 1..5: note = cleavable peptidic linker
IEPDT

SEQ ID NO: 128 - protein, 5 aa, synthetic construct
  REGION 1..5: note = cleavable peptidic linker
IEPDP

SEQ ID NO: 129 - protein, 5 aa, synthetic construct
  REGION 1..5: note = cleavable peptidic linker
LEPDG

SEQ ID NO: 130 - protein, 5 aa, synthetic construct
  REGION 1..5: note = cleavable peptidic linker
LEADG

SEQ ID NO: 131 - skipped

SEQ ID NO: 132 - protein, 4 aa, synthetic construct
  REGION 1..4: note = cleavable peptidic linker
RQAR

SEQ ID NO: 133 - protein, 5 aa, synthetic construct
  REGION 1..5: note = cleavable peptidic linker
RQARK

SEQ ID NO: 134 - skipped

SEQ ID NO: 135 - protein, 4 aa, synthetic construct
  REGION 1..4: note = cleavable peptidic linker
PAGL

SEQ ID NO: 136 - protein, 19 aa, synthetic construct
  REGION 1..19: note = cleavable peptidic linker
TGLEADGSPAGLGRQARVG

SEQ ID NO: 137 - protein, 19 aa, synthetic construct
  REGION 1..19: note = cleavable peptidic linker
TGLEADGSRQARVGPAGLG

SEQ ID NO: 138 - protein, 19 aa, synthetic construct
  REGION 1..19: note = cleavable peptidic linker
TGSPAGLEADGSRQARVGS

SEQ ID NO: 139 - protein, 19 aa, synthetic construct
  REGION 1..19: note = cleavable peptidic linker
TGPAGLGLEADGSRQARVG

SEQ ID NO: 140 - protein, 19 aa, synthetic construct
  REGION 1..19: note = cleavable peptidic linker
TGRQARVGLEADGSPAGLG

SEQ ID NO: 141 - protein, 18 aa, synthetic construct
  REGION 1..18: note = cleavable peptidic linker
TGSRQARVGPAGLEADGS

SEQ ID NO: 142 - protein, 20 aa, synthetic construct
  REGION 1..20: note = cleavable peptidic linker
TGPAGLGSRQARVGLEADGS

SEQ ID NO: 143 - protein, 18 aa, synthetic construct
  REGION 1..18: note = cleavable peptidic linker
GPAGLGLEPDGSRQARVG

SEQ ID NO: 144 - protein, 18 aa, synthetic construct
  REGION 1..18: note = cleavable peptidic linker
GGSGGGGIEPDIGGSGGS

SEQ ID NO: 145 - protein, 18 aa, synthetic construct
  REGION 1..18: note = cleavable peptidic linker
GGSGGGGLEADTGGSGGS

SEQ ID NO: 146 - protein, 9 aa, synthetic construct
  REGION 1..9: note = cleavable peptidic linker
GSIEPDIGS

SEQ ID NO: 147 - protein, 9 aa, synthetic construct
  REGION 1..9: note = cleavable peptidic linker
GSLEADTGS

SEQ ID NO: 148 - protein, 18 aa, synthetic construct
  REGION 1..18: note = cleavable peptidic linker
GGSGGGGIEPDGGGSGGS

SEQ ID NO: 149 - protein, 18 aa, synthetic construct
  REGION 1..18: note = cleavable peptidic linker
GGSGGGGIEPDVGGSGGS

SEQ ID NO: 150 - protein, 18 aa, synthetic construct
  REGION 1..18: note = cleavable peptidic linker
GGSGGGGIEPDSGGSGGS

SEQ ID NO: 151 - protein, 18 aa, synthetic construct
  REGION 1..18: note = cleavable peptidic linker
GGSGGGGIEPDTGGSGGS

SEQ ID NO: 152 - protein, 12 aa, synthetic construct
  REGION 1..12: note = cleavable peptidic linker
GGGSLEPDGSGS

SEQ ID NO: 153 - protein, 18 aa, synthetic construct
  REGION 1..18: note = cleavable peptidic linker
GPAGLGLEADGSRQARVG

SEQ ID NO: 154 - protein, 15 aa, synthetic construct
  REGION 1..15: note = cleavable peptidic linker
GGEGGGGSGGSGGGS

SEQ ID NO: 155 - protein, 18 aa, synthetic construct
  REGION 1..18: note = cleavable peptidic linker
GSSAGSEAGGSGQAGVGS

SEQ ID NO: 156 - protein, 18 aa, synthetic construct
  REGION 1..18: note = cleavable peptidic linker
GGSGGGGLEAEGSGGGGS

SEQ ID NO: 157 - protein, 18 aa, synthetic construct
  REGION 1..18: note = cleavable peptidic linker
GGSGGGGIEPDPGGSGGS

SEQ ID NO: 158 - protein, 19 aa, synthetic construct
  REGION 1..19: note = cleavable peptidic linker
TGGSGGGGIEPDIGGSGGS

SEQ ID NO: 159 - protein, 4 aa, synthetic construct
  REGION 1..4: note = elastase inhibitor
AAPV

SEQ ID NO: 160 - skipped

SEQ ID NO: 161 - protein, 13 aa, synthetic construct
  REGION 1..13: note = elastase inhibitor
KRCCPDTCGIKCL

SEQ ID NO: 162 - protein, 13 aa, synthetic construct
  REGION 1..13: note = elastase inhibitor
KRMMPDTMGIKML

SEQ ID NO: 163 - protein, 4 aa, synthetic construct
  REGION 1..4: note = cathepsin inhibitor
  SITE 1: note = N terminus protected by carboxybenzyl (=Z-group)
FFDK

SEQ ID NO: 164 - skipped

SEQ ID NO: 165 - protein, 65 aa, synthetic construct
  REGION 1..65: note = hirudin
MTYTDCTESGQNLCLCEGSNVCGQGNKCILGSDGEKNQCVTGEGTPKPQSHNDGDFEEIPEEYLQ

SEQ ID NO: 166 - protein, 4 aa, synthetic construct
  REGION 1..4: note = cleavable peptidic linker
PQAR

SEQ ID NO: 167 - protein, 5 aa, synthetic construct
  REGION 1..5: note = cleavable peptidic linker
PQARK

SEQ ID NO: 168 - protein, 4 aa, synthetic construct
  REGION 1..4: note = cleavable peptidic linker
HQAR

SEQ ID NO: 169 - protein, 5 aa, synthetic construct
  REGION 1..5: note = cleavable peptidic linker
HQARK

SEQ ID NO: 170 - skipped

SEQ ID NO: 171 - protein, 5 aa, synthetic construct
  REGION 1..5: note = cleavable peptidic linker
  SITE 5: note = MISC_FEATURE - X = A, V, or K
PMAKX

SEQ ID NO: 172 - protein, 4 aa, synthetic construct
  REGION 1..4: note = cleavable peptidic sequence
PMAK

SEQ ID NO: 173 - protein, 5 aa, synthetic construct
  REGION 1..5: note = cleavable peptidic linker
PMAKK

SEQ ID NO: 174 - DNA, 15 bp, synthetic construct
  misc_feature 1..15: note = GGGGS linker coding sequence
  source 1..15: mol_type = other DNA
ggcggcggcggcagc

SEQ ID NO: 175 - protein, 21 aa, Bos taurus
MKMSRLCLSVALLVLLGTLAA

SEQ ID NO: 176 - DNA, 63 bp, Bos taurus
  source 1..63: mol_type = genomic DNA
atgaagatgtctcgcctgtgccttagcgtggccctgctcgtcctgttgggaaccctggccgcc

SEQ ID NO: 177 - protein, 79 aa, Bos taurus
STPGCDTSNQAKAQRPDFCLEPPYTGPCKARMIRYFYNAKAGLCQPFVYGGCRAKRNNFKSSEDCMRTCGGAIGPWENL

SEQ ID NO: 178 - DNA, 237 bp, Bos taurus
  source 1..237: mol_type = genomic DNA
tccactcccggctgtgacacatccaaccaggcaaaggcccagcggcccgatttctgcctggaacctccatacaccggtccctgtaaagctaggatgatcagatatttttacaatgcaaaggccgggctgtgccaacctttcgtgtacggcggatgccgcgctaagcggaacaactttaaatcttccgaggactgtatgaggacttgcggtggcgccattgggccatgggaaaatctt

SEQ ID NO: 179 - protein, 451 aa, Cricetulus sp.
MAGRRLAQASISTVAVWLLCALRLQGTETALPSAPPELPGGAACLARFTSGVPAFVLDTDASVSNGATFLGSPTVRRSWDCVRACCTTQNCNLALVELQPDGGEDAISACFLMNCLYEQNFVCKFAPKEGFINYLTQELYHSYRELRSRGFGGSRIPRTWVGIDLKVQLQKPLVLRDADNTDWHLLEGDNDVRVERKSPDEVELWGLKEGTYMFQLTRTDSDQPEDTANLTITVLTAKQTEDYCLASYKVGRCRGSFPRWYYDPTEQICKSFTYGGCLGNKNNYLREEECMLACKDVQGISPKRHHPVCSGSCHATQFRCSNGCCIDGFLECDDTPDCPDGSDEATCDKYTSGFDELQSIHFTRDKGYCAELPDTGICKENIPRWYYNPFSEHCARFTYGGCYGNKNNFEEEQECLEACRGVSSERSGSGRRRMCLVCGGKSPFPMWAPWR

SEQ ID NO: 180 - DNA, 1356 bp, Cricetulus sp.
  source 1..1356: mol_type = genomic DNA
atggcaggcaggagactggctcaagccagcatctccaccgtggccgtctggctcctgtgcgctcttcgcctgcaagggacagagaccgcactcccctctgcccctcccgaactgcccggaggcgctgcatgtttggcccggttcactagcggcgtgcctgcttttgtcctggacacagatgccagcgtgtccaacggggcaaccttcctcggctctccaactgttaggagaagttgggactgcgtgcgcgcttgttgcaccacacagaattgtaacctggcccttgtcgagctgcagcccgatggaggcgaggacgccatttccgcctgctttttgatgaattgcctgtacgaacaaaacttcgtgtgcaagtttgcacctaaagagggcttcatcaactatctcacccaagagctgtaccactcctatcgggaacttaggtccagggggttcggaggctcaagaatcccacggacatgggtcggaatcgacctgaaggtgcagctccagaagcccctggttttgagggatgccgacaataccgattggcatctgctcgagggcgacaacgacgtcagagtcgagcgcaaaagccctgatgaagtggagctgtggggccttaaggaggggacttacatgtttcagctgacacgcacagactccgatcaaccagaagacactgctaatttgaccataacagtactgaccgcaaagcagacagaggactactgcctcgcctcttataaagtgggaaggtgtagaggcagtttcccccgctggtactatgatcccaccgagcagatctgcaagagctttacttacggcgggtgtctgggcaacaagaacaattaccttcgggaagaggagtgcatgctggcttgcaaagacgtccaggggatttcccctaagaggcaccacccagtttgtagcggaagttgccatgccacacaattcagatgtagcaacggctgctgtattgacggttttctcgagtgtgacgacacccccgattgccctgacgggtccgatgaggcaacttgtgacaagtatacctctggcttcgacgagctgcagtcaattcacttcacacgcgataaaggatactgcgctgaattgccagacaccggtatctgcaaggagaatataccccggtggtattacaaccctttttccgagcactgcgccaggttcacatacggcgggtgttatggaaacaagaataactttgaagaggaacaggagtgtctggaggcctgcagaggcgtgtcctctgagcgcagtggaagcggtcggagaagaatgtgtctcgtctgcggcgggaaatccccattccccatgtgggctccttggcgctga

(19) United States
(12) Patent Application Publication
(10) Pub. No.: US 2026/0098271 A1
(43) Pub. Date: Apr. 9, 2026

(54) METHOD FOR INCREASING RECOMBINANT PROTEIN EXPRESSION

(71) Applicant: Hoffmann-La Roche Inc., Little Falls, NJ (US)

(72) Inventors: Peter BRUENKER, Hittnau (CH); Martina GEIGER, Obfelden (CH); Simon AUSLAENDER, Wolfratshausen (DE); Ulrich GEOPFERT, Penzberg (DE)

(73) Assignee: Hoffmann-La Roche Inc., Little Falls, NJ (US)

(21) Appl. No.: 19/213,865

(22) Filed: May 20, 2025

Related U.S. Application Data
(63) Continuation of application No. PCT/EP2023/082474, filed on Nov. 21, 2023.

(30) Foreign Application Priority Data
Nov. 23, 2022 (EP) 22209191.0

(51) Int. Cl. C12N 15/67; C07K 14/47; C12N 9/10

(52) U.S. Cl. CPC C12N 15/67; C07K 14/4703; C12N 9/1003; C12N 2830/50

(57) ABSTRACT

Herein is reported a nucleic acid comprising in operably linked form a nucleic acid encoding a selection marker, a nucleic acid encoding a self-cleaving peptide sequence, and a nucleic acid encoding a proteinaceous protease inhibitor. Further reported are methods for the recombinant production of a heterologous polypeptide using said nucleic acid as well as a cell comprising said nucleic acid according to the invention. Likewise reported is the use of the nucleic acid according to the invention for increasing the amount of the recombinantly produced heterologous polypeptide by reducing protease cleavage.

### CROSS-REFERENCE TO RELATED APPLICATIONS

This application is a National Stage Entry of International Application No. PCT/EP2023/082474, filed Nov. 21, 2023, which claims benefit of and priority to European Patent Application No. 22209191.0, filed Nov. 23, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Sep. 19, 2025, is named P37882-US_SL.xml and is 205,361 bytes in size.

### FIELD OF THE INVENTION

The current invention is in the field of recombinant protein production. In more detail, the current invention is directed to the co-expression of a proteinaceous protease inhibitor, such as BPTI or aprotinin, in a mammalian cell recombinant for a heterologous polypeptide for increasing the amount of intact, i.e. not protease cleaved, recombinant heterologous polypeptide obtainable from the cultivation of said mammalian cell.

### BACKGROUND OF THE INVENTION

For the commercial production of therapeutic biologics, such as antibodies, one desirable property is that the expression yield of the therapeutic be maximized. The expression yield is depending directly on the production titer as well as indirectly on the amount of therapeutic-related side-products. These side-products can be, e.g., not correctly assembled therapeutic polypeptide, not correctly folded therapeutic polypeptide or degraded therapeutic polypeptide.

For the recombinant production of a therapeutic biologic generally mammalian cells, such as CHO and HEK cells, are used. Due to their origin, these cells also produce proteases, which can cleave the therapeutic. In case the therapeutic is amenable to cleavage by an endogenous protease of the producing cell, cleaved, i.e. degraded, therapeutic is obtained as a therapeutic-related side-product.

This problem has been addressed in different ways in the art.

In WO 99/10503 a process for the recombinant production of a serine protease using a recombinant nucleic acid which codes for a zymogenic precursor of the said serine protease wherein a naturally occurring non-autocatalytic cleavage site is replaced in the zymogenic precursor by an autocatalytic cleavage site which is recognized by the active form of the protease and thus cleaves the zymogenic precursor into the active form is reported.

In WO 02/61064 a method for the recombinant production of trypsin using a nucleic acid which codes for a trypsinogen with an enterokinase recognition site in the propeptide sequence in a form that can be secreted by the host cell and comprising the culturing of a cell comprising said nucleic acid under conditions which enable secretion of the expression product into the culture medium, whereby the conditions being selected such that an autocatalytic cleavage of the propeptide sequence is at least substantially prevented, is reported.

In CN111607615 a preparation method of a porcine epidemic diarrhea (PEDV) and porcine transmissible gastroenteritis (TGEV) virus disease bigeminal subunit vaccine using a PEDV-T2A-TGEV-S sequence and comprising the cultivating of insect cells comprising said sequence in a culture medium comprising a mixture of the serine protease inhibitor aprotinin, the aspartic protease inhibitor pepstatin a, the aminopeptidase inhibitor bestatin, the cysteine protease inhibitor E-64 and the serine/cystine protease inhibitor leupeptin for 24 hours to obtain a culture containing the protease inhibitor, and continuously culturing the culture containing the protease inhibitor for 48 hours to obtain a culture containing an expressed protein, is reported.

In WO 2016/118775 a method of producing a retroviral producer cell that stably produces a cocal envelope pseudotyped retroviral vector comprising transfecting a cell with a plasmid encoding a cocal envelope, a self-cleaving peptide, and a selection marker, is reported.

In WO 2021/183946 an expression vector comprising a nucleic acid comprising an open reading frame that encodes a protein of interest followed by a self-cleaving peptide, which can induce ribosomal skipping during translation, and a selectable marker gene, is reported.

In CN110835632 an expression cassette comprising a promoter, a framework sequence of a sgRNA for a mutant Cas9 derived from Saccharomyces cerevisiae, the human uracil glycosidase inhibitor, the T2A self-splicing polypeptide, green fluorescent protein and a BGH polyA sequence, is reported.

In CN 108441516 the lentivirus CMV-CBh double promoter modified vector pLenti-CMV-3 FLAG-EGFP-PGK-mCherry-T2A-Puro is reported.

In CN 112626121 a three-selectable marker antibody expression vector comprising an expression frame I and an expression frame II, wherein the expression frame II is adjacent to the expression frame I in a forward arrangement mode, wherein in expression frame I, a puromycin resistance gene coupled at the downstream of a light chain gene of an antibody through an IRES sequence, and then mouse glutamine synthetase is coupled at the downstream of the puromycin resistance gene through E2A polypeptide, wherein in expression frame II the dihydrofolate reductase gene DHFR coupled downstream of the antibody heavy chain gene by IRES sequence, is reported.

In WO 2019/157099 a nasal spray pharmaceutical formulation comprising epinephrine, or a salt thereof, and comprising one or more absorption enhancers, such as aprotinin, is reported.

In WO 2019/239405 an oral pharmaceutical composition comprising a therapeutic peptide or therapeutic protein of up to 100 kilodaltons, a chelator of divalent cations, and an isolated recombinantly expressed Bowman-Birk inhibitor (BBI), wherein the recombinantly expressed BBI is expressed in a Pichia pastoris expression system, is reported.

In WO 2008/005847 it is reported that FVIII may be expressed in media containing a protease inhibitor such as aprotinin, e.g. in an amount from about 0.01 to about 5%, or from about 0.5 to about 1.0%, (vol/vol) (Aprot., 15-30 Trypsin inhibitor units (TIU)/ml, Sigma) or corresponding amounts of activity units of other protease inhibitors.

In WO 2008/135501 a method for the production of a Factor VIII polypeptide by culturing a mammalian cell expressing a Factor VIII polypeptide, wherein the cell culture medium comprises a soybean trypsin inhibitor, is reported.

In WO 2018/22032 a method for the recombinant production of debrilase comprising the culturing of a host cell comprising one or more vectors, wherein the one or more vectors encode in expressible form debrilase and a proteinaceous inhibitor of debrilase under conditions wherein debrilase and the proteinaceous inhibitor of debrilase are expressed, wherein the proteinaceous inhibitor of debrilase is selected from the group consisting of aprotinin, plasminostreptin (I16.001), proteinase inhibitor type-2 K (PIN2K, I20.001), ecotin (I11.001), trypsin inhibitor MCTI-1 (I07.001), ascidian trypsin inhibitor (I05.001), peptidase inhibitor 5 (KappaPI-actitoxin-Avd3a, I02.026), and tissue factor pathway inhibitor-2 inhibitor unit 1 (TFPI2, I02.013), is reported.

In WO 2021/170839 the use of aprotinin as a carrier to produce a recombinant protein, polypeptide or peptide in algae, wherein aprotinin and said recombinant protein, polypeptide or peptide are fused together to form a fusion protein, is reported.

Thus, there is the need for improved methods for the recombinant production of therapeutic polypeptides.

### SUMMARY OF THE INVENTION

Herein is reported a method for the production of a heterologous polypeptide by a recombinant mammalian cell, wherein the heterologous polypeptide is susceptible to cleavage by an endogenous protease produced by said recombinant mammalian cell, whereby the cleavage of said heterologous polypeptide by said endogenous protease is reduced by the co-expression of a proteinaceous protease inhibitor.

Thus, herein is reported a nucleic acid comprising a first part encoding a selection marker and a second part encoding a proteinaceous protease inhibitor whereby both parts are linked by an internal ribosomal entry site (IRES) or an intein or a self-cleaving peptide sequence. Likewise reported herein is the use of the nucleic acid according to the invention in the recombinant production of a heterologous polypeptide as well as a cell comprising said nucleic acid according to the invention. Further reported herein is the use of the nucleic acid according to the invention for increasing the production yield of a recombinantly produced heterologous polypeptide as well as its use for reducing protease cleavage of a recombinant heterologous polypeptide during production in a mammalian cell.

The invention is based, at least in part, on the finding that the combination of a selectable marker and a proteinaceous protease inhibitor in a single, i.e. the same, cistron, e.g. using an IRES or an intein or a self-cleaving peptide sequence, is advantageous and provides for improvements. The improvements are, amongst others, at least the reduction of protease cleavage during recombinant production (expression) of a heterologous polypeptide in a mammalian cell and thereby an increased production amount (yield). At the same time the expression of the recombinant heterologous polypeptide is not reduced compared to a cell not comprising the nucleic acid according to the current invention.

The invention is based, at least in part, on the finding that a standard co-expression of a proteinaceous protease inhibitor and a recombinant heterologous polypeptide reduces the amount (yield) of the heterologous polypeptide compared to a cell not expressing the proteinaceous protease inhibitor.

The invention is based, at least in part, on the finding that the knock-out of a single protease or of all variants of a single protease in a cell expressing a heterologous polypeptide does not result in the same increase of the amount (yield) of the recombinant heterologous polypeptide as the use of the nucleic acid according to the current invention. Without being bound by this theory, it is assumed that in case cleavage is observed during recombinant production of a heterologous polypeptide, it is often caused by more than one protease.

The invention is further based, at least in part, on the finding that the promoter for driving the expression of the nucleic acid according to the invention shall not be a strong promoter, i.e. it shall not be, e.g., the CMV promoter, the human EF1a promoter or the CAG promoter. It has been found that it is advantageous to use a promoter with intermediate strength, such as, e.g., the SV40 promoter, the PGK1 promoter or the Ubc promoter, whereby the SV40 promoter is one preferred promoter.

It has to be pointed out that the term “a self-cleaving peptide sequence” in the following aspects and embodiments can be replaced by the term “an IRES” or by the term “intein” without departing from the invention. These are also specific aspects and embodiments of the current invention.

An IRES element when placed between two genes mediates cap-independent translation of the second gene.

Inteins are generally flanked by the two genes and translated together in a single polypeptide before splicing occurs to remove the intein to join the flanking proteins with a new bond. Mutations to the intein sequence inhibit the splicing while preserving the cleavage.

One aspect of the invention is a (isolated) nucleic acid comprising in 5′- to 3′- or in 3′- to 5′-direction in operably linked form the following elements
a) a nucleic acid encoding a selection marker,
b) a nucleic acid encoding a self-cleaving peptide sequence, and
c) a nucleic acid encoding a proteinaceous protease inhibitor.

In certain embodiments of all aspects and embodiments, the nucleic acid further comprises in operably linked form the following elements
d) a promoter upstream (5′) to the nucleic acid of a) (5′- to 3′-direction) or c) (3′- to 5′-direction),
e) a polyadenylation signal sequence downstream (3′) to the nucleic acid of c) (5′- to 3′-direction) or a) (3′- to 5′-direction), and
f) optionally a terminator sequence downstream (3′) to the nucleic acid of e).

In certain embodiments of all aspects and embodiments, the nucleic acid encoding a selection marker is selected from the group consisting of nucleic acids encoding puromycin acetyltransferase, histidinol dehydrogenase, thymidine kinase, hygromycin B phosphotransferase, dihydrofolate reductase, blasticidin deaminase, glutamine synthase, zeocin resistance gene, bleomycin resistance gene, and aminoglycoside 3′ phosphotransferase.

In certain embodiments of all aspects and embodiments, the nucleic acid encoding a selection marker encodes puromycin acetyltransferase or a functional variant thereof that is capable of inactivating/modifying puromycin.

In certain embodiments of all aspects and embodiments, the nucleic acid encoding a selection marker encodes the amino acid sequence of SEQ ID NO: 01 (SMAA; Uniprot P13249 (PUAC_STRAD) excluding the N-terminal methionine residue) or a functional variant thereof that is capable of inactivating/modifying puromycin.

In certain embodiments of all aspects and embodiments,
the nucleic acid encoding a selection marker has the nucleotide sequence of SEQ ID NO: 02 (SMNN), or
the nucleic acid encoding a selection marker is a variant of the nucleotide sequence of SEQ ID NO: 02 encoding a selection marker that has the amino acid sequence of SEQ ID NO: 01, or
the nucleic acid encoding a selection marker encodes a functional variant of SEQ ID NO: 01 that is capable of inactivating/modifying puromycin.

In certain embodiments of all aspects and embodiments, the self-cleaving peptide sequence is a viral 2-A self-cleaving peptide sequence or a functional variant thereof that is capable of effecting ribosome skipping.

In certain embodiments of all aspects and embodiments, the self-cleaving peptide sequence is selected from the group consisting of P2A, T2A, F2A, E2A, A2A, D2A, I2A, the self-cleaving peptides of SEQ ID NO: 64, 66, 68, 70, 72, 74, 76, 78, 80, 82, 84, or functional variants thereof.

In certain embodiments of all aspects and embodiments, the self-cleaving peptide sequence is T2A or a functional variant thereof that is capable of effecting ribosome skipping.

In certain embodiments of all aspects and embodiments, the nucleic acid encoding a self-cleaving peptide sequence encodes the amino acid sequence of SEQ ID NO: 14 (SCAA) or a functional variant thereof that is capable of effecting ribosome skipping.

In certain embodiments of all aspects and embodiments,
the nucleic acid encoding a self-cleaving peptide sequence has the nucleotide sequence of SEQ ID NO: 15 (SCNN), or
the nucleic acid encoding a self-cleaving peptide sequence is a variant of the nucleotide sequence of SEQ ID NO: 15 (SCNN) encoding a self-cleaving peptide sequence that has the amino acid sequence of SEQ ID NO: 14 (SCAA), or
the nucleic acid encoding a self-cleaving peptide sequence encodes a functional variant of SEQ ID NO: 14 (SCAA) that is capable of effecting ribosome skipping.

In certain embodiments of all aspects and embodiments, the proteinaceous protease inhibitor inhibits one or more proteases selected from the group of proteases consisting of ADAMS, ADAMTS, e.g. ADAM8; ADAM9; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKs, e.g., KLK4, KLK5, KLK6, KLK7, KLK8, KLK10, KLK11, KLK13, or KLK14; Metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, and TMPRSS4.

In certain embodiments of all aspects and embodiments, the nucleic acid encoding a proteinaceous protease inhibitor is selected from the group consisting of BPTI, aprotinin, leupeptin (LLR), pepstatin (iVVVStaASta), SPINT1, SPINK1, HAI-1, HAI-2 or a functional variant thereof.

In certain embodiments of all aspects and embodiments, the nucleic acid encoding a proteinaceous protease inhibitor encodes BPTI or a functional variant thereof that is capable of inhibiting one or more serine proteases.

In certain embodiments of all aspects and embodiments, the nucleic acid encoding a proteinaceous protease inhibitor encodes the amino acid sequence of SEQ ID NO: 86 (AprotAA) or a functional variant thereof that is capable of inhibiting one or more serine proteases.

In certain embodiments of all aspects and embodiments,
the nucleic acid encoding a proteinaceous protease inhibitor has the nucleotide sequence of SEQ ID NO: 87 (AprotNN), or
the nucleic acid encoding a proteinaceous protease inhibitor is a variant of the nucleotide sequence of SEQ ID NO: 87 (AprotNN) encoding a proteinaceous protease inhibitor that has the amino acid sequence of SEQ ID NO: 86 (AprotAA), or
the nucleic acid encoding a proteinaceous protease inhibitor encodes a functional variant of SEQ ID NO: 86 (AprotAA) that is capable of inhibiting one or more serine proteases.

In certain embodiments of all aspects and embodiments, the promoter is the SV40 (simian virus 40) promoter or a functional variant thereof with the same (comparable) or lesser promoter strength.

In certain embodiments of all aspects and embodiments, the promoter has the nucleotide sequence of SEQ ID NO: 102 (PromNN) or is a functional variant thereof that has the same or lesser promoter strength.

In certain embodiments of all aspects and embodiments, the polyadenylation signal sequence is the bGH (bovine growth hormone) polyadenylation signal sequence.

In certain embodiments of all aspects and embodiments, the polyadenylation signal sequence has the nucleotide sequence of SEQ ID NO: 100 (PolyANN).

In certain embodiments of all aspects and embodiments, the terminator sequence is present and is the hGT (human growth hormone terminator) sequence.

In certain embodiments of all aspects and embodiments, the terminator sequence is present and has the nucleotide sequence of SEQ ID NO: 101 (TermNN).

One aspect of the invention is a (recombinant) cell comprising the nucleic acid according to the invention.

In certain embodiments of all aspects and embodiments, the nucleic acid according to the invention is stably integrated into the genome of the cell/into a chromosome of the cell.

In certain embodiments of all aspects and embodiments, a single copy of the nucleic acid according to the invention is integrated into the genome of the cell/into a chromosome of the cell.

In certain embodiments of all aspects and embodiments, the cell further comprises one or more nucleic acid sequences encoding a (recombinant) heterologous polypeptide.

In certain embodiments of all aspects and embodiments, the one or more nucleic acid sequences encoding a (recombinant) heterologous polypeptide are stably integrated into the genome of the cell/into a chromosome of the cell.

In certain embodiments of all aspects and embodiments, the one or more nucleic acid sequences encoding a (recombinant) heterologous polypeptide are integrated at a single site into the genome of the cell/into a single site in one chromosome of the cell.

In certain embodiments of all aspects and embodiments, the nucleic acid according to the invention and the one or more nucleic acid sequences encoding a (recombinant) heterologous polypeptide are integrated at the same site into the genome of the cell/into a chromosome of the cell.

In certain embodiments of all aspects and embodiments, the cell further comprises one or more nucleic acid sequences encoding a (recombinant) heterologous polypeptide that comprises one or more protease-cleavable amino acid sequences/protease recognition sequences.

In certain embodiments of all aspects and embodiments, the cell further comprises one or more nucleic acid sequences encoding a (recombinant) heterologous polypeptide that comprises one or more amino acid sequences cleavable by a serine protease/serine protease recognition sequences.

In certain embodiments of all aspects and embodiments, the cell further comprises one or more nucleic acid sequences encoding a (recombinant) heterologous polypeptide that comprises one or more amino acid sequences cleavable by a protease selected from the group of proteases consisting of ADAMS, ADAMTS, e.g. ADAM8; ADAM9; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKS, e.g., KLK4, KLK5, KLK6, KLK7, KLK8, KLK10, KLK11, KLK13, or KLK14; Metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4; and any combination thereof.

In certain embodiments of all aspects and embodiments, the recombinant heterologous polypeptide is an antibody that comprises one or more protease-cleavable amino acid sequences/protease recognition sequences.

In certain embodiments of all aspects and embodiments, the cell is a mammalian cell.

In certain embodiments of all aspects and embodiments, the cell is a CHO cell or a HEK cell or a BHK cell.

In certain embodiments of all aspects and embodiments, the cell is a CHO-K1 cell.

One aspect of the invention is a method for producing a (recombinant) heterologous polypeptide, wherein the method comprises the following steps:
cultivating a cell according to the invention in a cultivation medium to produce the (recombinant) heterologous polypeptide,
recovering the (recombinant) heterologous polypeptide from the cell or the cultivation medium, and
optionally purifying the (recombinant) heterologous polypeptide by one or more chromatography steps.

In certain embodiments of all aspects and embodiments, the cultivating is (at least for some time/days) in the presence of puromycin, histidinol, ganciclovir, hygromycin, methionine sulphoximine, blasticidin, methotrexate, zeocin, bleomycin, or neomycin (G418).

In certain embodiments of all aspects and embodiments, the cultivating is (at least for some time/days) in the presence of puromycin or a functional variant thereof.

In certain embodiments of all aspects and embodiments, the amount of recovered not-cleaved (recombinant) heterologous polypeptide is increased compared to a method wherein a cell not comprising a nucleic acid according to the invention is used.

One aspect of the invention is the use of a nucleic acid according to the invention for reducing protease cleavage of a (recombinant) heterologous polypeptide during recombinant production in a mammalian cell.

One aspect of the invention is the use of a nucleic acid according to the invention for increasing the amount of recovered intact/not-protease cleaved (recombinant) heterologous polypeptide from the cultivation of a mammalian cell.

The current invention encompasses at least the following independent aspects and dependent embodiments:
1. A nucleic acid comprising in operably linked form the following elements
   a) a nucleic acid encoding a selection marker,
   b) a nucleic acid encoding a self-cleaving peptide sequence, and
   c) a nucleic acid encoding a proteinaceous protease inhibitor.
2. The nucleic acid according to embodiment 1, wherein the elements in 5′- to 3′-direction have the sequence of a)-b)-c).
3. The nucleic acid according to embodiment 1, wherein the elements in 5′- to 3′-direction have the sequence of c)-b)-a).
4. The nucleic acid according to any one of embodiments 1 to 3, wherein the nucleic acid further comprises in operably linked form the following elements
   d) a promoter upstream (5′) to the first element,
   e) a polyadenylation signal sequence downstream (3′) to the last element, and f) optionally a terminator sequence downstream (3′) to the nucleic acid of e).
5. The nucleic acid according to any one of embodiments 1 to 4, wherein the nucleic acid encoding a selection marker is selected from the group consisting of nucleic acids encoding puromycin acetyltransferase, histidinol dehydrogenase, thymidine kinase, hygromycin B phosphotransferase, dihydrofolate reductase, blasticidin deaminase, glutamine synthase, zeocin resistance gene, bleomycin resistance gene, and aminoglycoside 3′ phosphotransferase.
6. The nucleic acid according to any one of embodiments 1 to 5, wherein the nucleic acid encoding a selection marker encodes puromycin acetyltransferase or a functional variant thereof capable of inactivating/modifying puromycin.
7. The nucleic acid according to any one of embodiments 1 to 6, wherein the nucleic acid encoding a selection marker encodes the amino acid sequence of SEQ ID NO: 01 or a functional variant thereof capable of inactivating/modifying puromycin.
8. The nucleic acid according to any one of embodiments 1 to 7, wherein
   the nucleic acid encoding a selection marker has the nucleotide sequence of SEQ ID NO: 02, or
   the nucleic acid encoding a selection marker is a variant of the nucleotide sequence of SEQ ID NO: 02 encoding a selection marker that has the amino acid sequence of SEQ ID NO: 01, or
   the nucleic acid encoding a selection marker encodes a functional variant of SEQ ID NO: 01 capable of inactivating/modifying puromycin.
9. The nucleic acid according to any one of embodiments 1 to 8, wherein the nucleic acid encoding a selection marker encodes the amino acid sequence of SEQ ID NO: 01.
10. The nucleic acid according to any one of embodiments 1 to 9, wherein the self-cleaving peptide sequence is a viral 2-A self-cleaving peptide sequence or a functional variant thereof capable of effecting ribosome skipping.
11. The nucleic acid according to any one of embodiments 1 to 10, wherein the self-cleaving peptide sequence is selected from the group consisting of P2A, T2A, F2A, E2A, A2A, D2A, I2A, the self-cleaving peptides of SEQ ID NO: 64, 66, 68, 70, 72, 74, 76, 78, 80, 82, 84 or a functional variant thereof.
12. The nucleic acid according to any one of embodiments 1 to 11, wherein the self-cleaving peptide sequence is T2A or a functional variant thereof capable of effecting ribosome skipping.
13. The nucleic acid according to any one of embodiments 1 to 12, wherein the self-cleaving peptide sequence is T2A.
14. The nucleic acid according to any one of embodiments 1 to 13, wherein the nucleic acid encoding a self-cleaving peptide sequence encodes the amino acid sequence of SEQ ID NO: 14 or a functional variant thereof capable of effecting ribosome skipping.
15. The nucleic acid according to any one of embodiments 1 to 14, wherein
   the nucleic acid encoding a self-cleaving peptide sequence has the nucleotide sequence of SEQ ID NO: 15, or
   the nucleic acid encoding a self-cleaving peptide sequence is a variant of the nucleotide sequence of SEQ ID NO: 15 encoding a self-cleaving peptide sequence that has the amino acid sequence of SEQ ID NO: 14, or
   the nucleic acid encoding a self-cleaving peptide sequence encodes a functional variant of SEQ ID NO: 14 capable of effecting ribosome skipping.
16. The nucleic acid according to any one of embodiments 1 to 15, wherein the nucleic acid encoding a self-cleaving peptide sequence encodes the amino acid sequence of SEQ ID NO: 14.
17. The nucleic acid according to any one of embodiments 1 to 16, wherein the proteinaceous protease inhibitor inhibits one or more proteases selected from the group of proteases consisting of ADAMS, ADAMTS, e.g. ADAM8; ADAM9; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKs, e.g., KLK4, KLK5, KLK6, KLK7, KLK8, KLK10, KLK11, KLK13, or KLK14; Metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, and TMPRSS4.
18. The nucleic acid according to any one of embodiments 1 to 17, wherein the nucleic acid encoding a proteinaceous protease inhibitor is selected from the group consisting of BPTI, aprotinin, leupeptin (LLR), pepstatin (iVVVStaASta), SPINT1, SPINK1, HAI-1, HAI-2 or a functional variant thereof.
19. The nucleic acid according to any one of embodiments 1 to 18, wherein the nucleic acid encoding a proteinaceous protease inhibitor encodes BPTI or a functional variant thereof capable of inhibiting one or more serine proteases.
20. The nucleic acid according to any one of embodiments 1 to 19, wherein the nucleic acid encoding a proteinaceous protease inhibitor encodes BPTI.
21. The nucleic acid according to any one of embodiments 1 to 20, wherein the nucleic acid encoding a proteinaceous protease inhibitor encodes the amino acid sequence of SEQ ID NO: 86 or SEQ ID NO: 177 or a functional variant thereof capable of inhibiting one or more serine proteases.
22. The nucleic acid according to any one of embodiments 1 to 21, wherein
   the nucleic acid encoding a proteinaceous protease inhibitor has the nucleotide sequence of SEQ ID NO: 87 or SEQ ID NO: 178, or
   the nucleic acid encoding a proteinaceous protease inhibitor is a variant of the nucleotide sequence of SEQ ID NO: 87 encoding a proteinaceous protease inhibitor that has the amino acid sequence of SEQ ID NO: 86, or
   the nucleic acid encoding a proteinaceous protease inhibitor encodes a functional variant of SEQ ID NO: 86 capable of inhibiting one or more serine proteases.
23. The nucleic acid according to any one of embodiments 1 to 22, wherein the nucleic acid encoding a proteinaceous protease inhibitor encodes the amino acid sequence of SEQ ID NO: 86.
24. The nucleic acid according to any one of embodiments 4 to 23, wherein the promoter is the SV40 promoter or a functional variant thereof with the same or lesser promoter strength.
25. The nucleic acid according to any one of embodiments 4 to 24, wherein the promoter is the SV40 promoter.
26. The nucleic acid according to any one of embodiments 4 to 25, wherein the promoter has the nucleotide sequence of SEQ ID NO: 102.
27. The nucleic acid according to any one of embodiments 4 to 26, wherein the polyadenylation signal sequence is the bGH polyadenylation signal sequence.
28. The nucleic acid according to any one of embodiments 4 to 27, wherein the polyadenylation signal sequence has the nucleotide sequence of SEQ ID NO: 100.
29. The nucleic acid according to any one of embodiments 4 to 28, wherein the terminator sequence is present and is the hGT terminator sequence.
30. The nucleic acid according to any one of embodiments 4 to 29, wherein the terminator sequence is present and has the nucleotide sequence of SEQ ID NO: 101.
31. A vector comprising the nucleic acid according to any one of embodiments 1 to 30 and optionally further regulatory elements.
32. A cell comprising the nucleic acid according to any one of embodiments 1 to 30 or the vector according to embodiment 31.
33. The cell according to embodiment 32, wherein the nucleic acid is stably integrated into the genome of the cell/into a chromosome of the cell.
34. The cell according to any one of embodiments 32 to 33, wherein a single copy of the nucleic acid is integrated into the genome of the cell/into a chromosome of the cell.
35. The cell according to any one of embodiments 32 to 34, wherein the cell further comprises one or more nucleic acid sequences encoding a heterologous polypeptide.
36. The cell according to embodiment 35, wherein the one or more nucleic acid sequences encoding a heterologous polypeptide are stably integrated into the genome of the cell/into a chromosome of the cell.
37. The cell according to any one of embodiments 35 to 36, wherein the one or more nucleic acid sequences encoding a heterologous polypeptide are integrated at a single site into the genome of the cell/into a single site in one chromosome of the cell.
38. The cell according to any one of embodiments 35 to 37, wherein the nucleic acid according to any one of embodiments 1 to 30 or the plasmid according to embodiment 31 and the one or more nucleic acid sequences encoding a heterologous polypeptide are integrated at the same site into the genome of the cell/into a chromosome of the cell.
39. The cell according to any one of embodiments 35 to 38, wherein the heterologous polypeptide comprises one or more protease-cleavable amino acid sequences/protease recognition sequences.
40. The cell according to any one of embodiments 35 to 39, wherein the heterologous polypeptide comprises one or more amino acid sequences cleavable by a serine protease/serine protease recognition sequences.
41. The cell according to any one of embodiments 32 to 40, wherein the cell further comprises one or more nucleic acid sequences encoding a recombinant heterologous polypeptide that comprises one or more amino acid sequences cleavable by a protease selected from the group of proteases consisting of ADAMS, ADAMTS, e.g. ADAM8; ADAM9; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKs, e.g., KLK4, KLK5, KLK6, KLK7, KLK8, KLK10, KLK11, KLK13, or KLK14; Metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4; and any combination thereof.
42. The cell according to any one of embodiments 35 to 41, wherein the heterologous polypeptide is an antibody that comprises one or more protease-cleavable amino acid sequences.
43. The cell according to any one of embodiments 32 to 42, wherein the cell is a mammalian cell.
44. The cell according to any one of embodiments 32 to 43, wherein the cell is a CHO cell or a HEK cell or a BHK cell.
45. The cell according to any one of embodiments 32 to 44, wherein the cell is a CHO-K1 cell.
46. A method for producing a heterologous polypeptide in a recombinant cell, wherein the method comprises the following steps:
   cultivating a cell according to any one of embodiments 35 to 45 in a cultivation medium to produce the heterologous polypeptide,
   recovering the heterologous polypeptide from the cell or the cultivation medium, and
   optionally purifying the heterologous polypeptide by one or more chromatography steps.
47. The method according to embodiment 46, wherein the cultivating is in the presence of puromycin, histidinol, ganciclovir, hygromycin, methionine sulphoximine, blasticidin, methotrexate, zeocin, bleomycin, or neomycin (G418).
48. The method acid according to any one of embodiments 46 to 47, wherein the cultivating is in the presence of puromycin or a functional variant thereof.
49. The method acid according to any one of embodiments 46 to 48, wherein the cultivating is in the presence of puromycin.
50. The method according to any one of embodiments 46 to 49, wherein the amount of recovered not-cleaved heterologous polypeptide is increased compared to a method wherein a cell not comprising a nucleic acid according to any one of embodiments 1 to 30 or a vector according to embodiment 31 is used.
51. Use of a nucleic acid according to any one of embodiments 1 to 30 or a vector according to embodiment 31 for reducing protease cleavage of a heterologous polypeptide during recombinant production in a mammalian cell.
52. Use of a nucleic acid according to any one of embodiments 1 to 30 or a vector according to embodiment 31 for increasing the amount of recovered intact/not-protease cleaved heterologous polypeptide from the cultivation of a mammalian cell recombinant for said heterologous polypeptide.

In addition to the various aspects and embodiments depicted and claimed herein, the disclosed subject matter is also directed to other aspects and embodiments having other combinations of the features disclosed and claimed herein. As such, the particular features presented herein, especially presented as aspects or embodiments, can be combined with each other in other manners within the scope of the disclosed subject matter such that the disclosed subject matter includes any suitable combination of the features disclosed herein. The description of specific embodiments of the disclosed subject matter has been presented for purposes of illustration and description. It is not intended to be exhaustive or to limit the disclosed subject matter to those embodiments disclosed.

### DESCRIPTION OF THE FIGURES

FIG. 1 Composition of the product obtained from (a) a cell line expressing an exemplary heterologous protein in the absence of BPTI compared to (b) a cell line co-expressing BPTI, as measured by CE-SDS.

FIG. 2 Percentages of intact vs. cleaved main product produced in different cultivations; cell without inhibitor (circle); co-expression of one protease-specific inhibitor (star); knock-out of one specific protease (one variant individually (light) or combined (dark)) (crosses).

FIG. 3 Supernatant product concentration of a cultivation of a cell in the absence of a protease inhibitor and of a cultivation with co-expression of a soluble protease-specific inhibitor.

FIG. 4 Total product concentration (sum of uncleaved and cleaved main product) of a cultivation of a cell without protease inhibitor (triangle) and of a cultivation with co-expression of a soluble protease-specific inhibitor employing the medium strength SV40 promoter (circle) versus the percentage of main product.

FIG. 5 Total product concentration (sum of uncleaved and cleaved main product) versus percentage of uncleaved product of a cultivation of a cell without protease inhibitor and a cultivation with co-expression of a soluble protease-specific inhibitor employing the medium strength SV40 promoter (triangle: cell without inhibitor; circle: cell with co-expression of protease inhibitor driven by medium strength SV40 promoter).

FIG. 6 Total product concentration of a cultivation of a cell with co-expression of a soluble protease-specific inhibitor and a cultivation with the addition of the proteinaceous serine protease inhibitor BPTI to the cultivation medium (=sum of cleaved and uncleaved product) (triangle: cell without inhibitor; circle: cell with addition of BPTI) versus the percentage of total main product (sum of uncleaved and cleaved main product).

FIG. 7 Total product concentration (sum of uncleaved and cleaved main product) versus percentage of uncleaved product of a cultivation of a cell with co-expression of a soluble protease-specific inhibitor and a cultivation with the addition of the proteinaceous serine protease inhibitor BPTI to the cultivation medium (triangle: cell without inhibitor; circle: cell with addition of BPTI).

FIG. 8 Titer (as determined by protein A chromatography) and relative amounts of cleaved and uncleaved product (as determined by reducing CE-SDS) for different cultivation conditions.

### DETAILED DESCRIPTION OF THE INVENTION

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term “about” denotes a range of +/−10% of the thereafter following numerical value. In certain embodiments, the term “about” denotes a range of +/−5% of the thereafter following numerical value.

The term “comprising” also encompasses the term “consisting of”.

### The Nucleic Acid According to the Current Invention

The current invention is based at least in part on the finding that the combination of a selectable marker and a proteinaceous protease inhibitor in a single cistron, e.g. using a self-cleaving peptide sequence or an IRES for linking, is advantageous and provides improvements. The improvements are, amongst others, the reduction of protease cleavage during recombinant production (expression) of a heterologous polypeptide in a mammalian cell and thereby increasing the production amount (yield). At the same time the expression titer of the recombinant heterologous polypeptide is not reduced compared to a cell not comprising the nucleic acid according to the current invention.

It has been found that during recombinant production of a polypeptide that comprises one or more amino acid sequences cleavable by a serine protease, i.e. that comprises one or more serine protease recognition (amino acid) sequences, the cleavage of said sequences already occurs.

The invention is exemplified in the following using an N-terminal Fab-domain inserted 2+1 bispecific antibody (TCB) with an additional functional group connected by a peptidic linker that comprises one or more amino acid sequences cleavable by a serine protease/serine protease recognition sequences. This is presented solely as an exemplification of the method according to the current invention and shall not be construed as limitation thereof. The true scope is set forth in the appended claims.

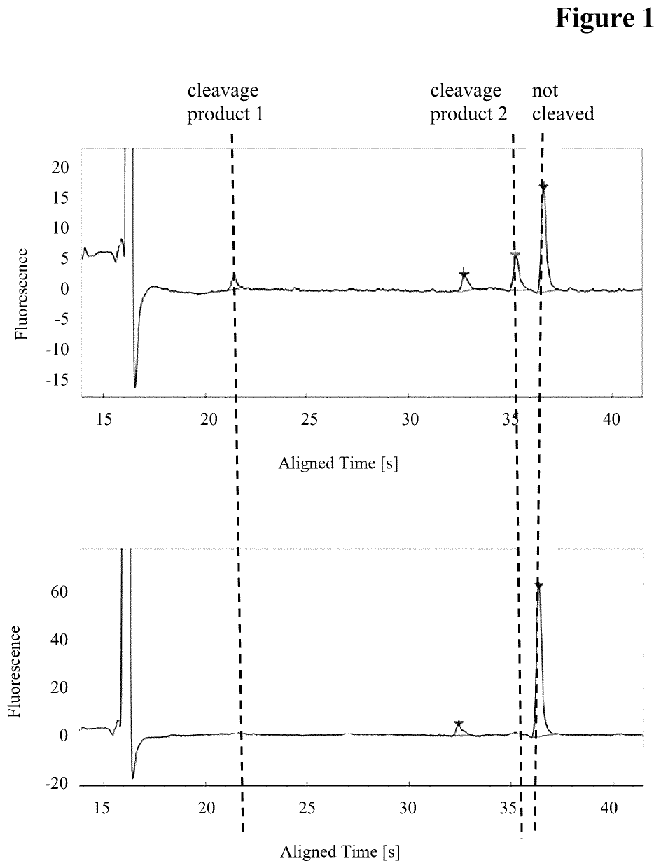

Cleavage occurs independent of the cell expressing the heterologous polypeptide. The extent of cleavage for different cells used for expression is shown in the following Table 1 and in FIG. 1.

TABLE 1
Cleavage dependent on the recombinant cell.

| Cell | cleaved product of total product |
|---|---|
| ExpiCHO-S | 3% |
| HEK293 | 9% |
| CHO-K1 | 34% |

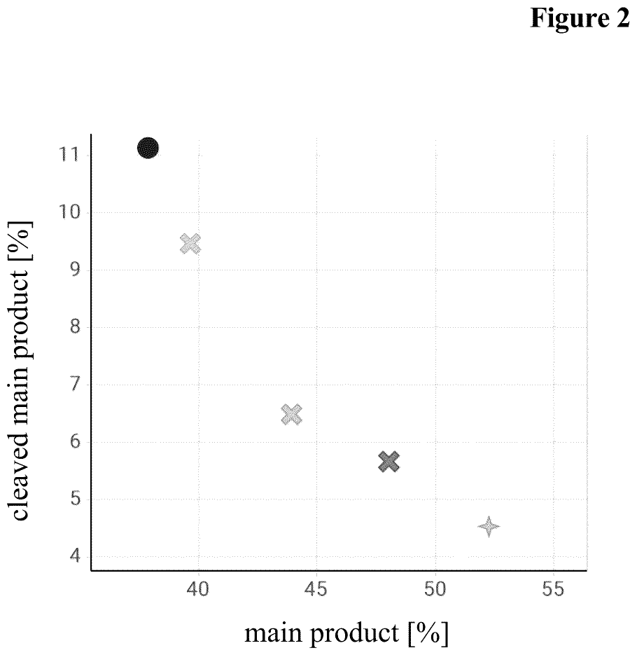

Generally, the cleavage of the recombinant polypeptide during production cannot be attributed to a single protease of the cell. Thus, it was expected that by reducing the activity of one single protease by co-expressing a soluble protease-specific inhibitor the amount of cleaved recombinant polypeptide cannot be reduced significantly. However, by co-expressing one protease-specific inhibitor the fraction of intact, i.e. not-cleaved recombinant polypeptide could be increased from about 38% (circle in FIG. 2, x-axis) to more than 50% (star in FIG. 2, x-axis) and at the same time the fraction of cleaved recombinant polypeptide could be reduced from about 11% (circle in FIG. 2, y-axis) to about 4.5% (star in FIG. 2, y-axis). For comparison the knock-out of the respective protease (one variant individually each or both combined combined) in the respective cell pool is shown. The knock-out resulted in a cell population with heterogeneous knock-out (e.g. homozygous, heterozygous, no knockout), which resulted in intermediate cleavage levels (crosses in FIG. 2).

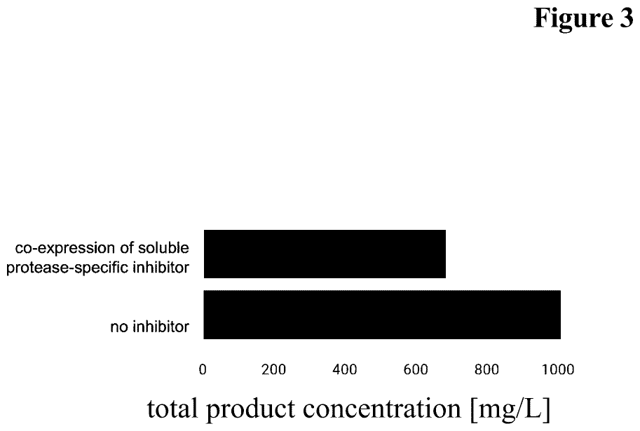

However, at the same time the total polypeptide titer was dramatically reduced by about 30% from 1000 mg/L to 700 mg/L so that overall no improvement in yield could be achieved (see FIG. 3).

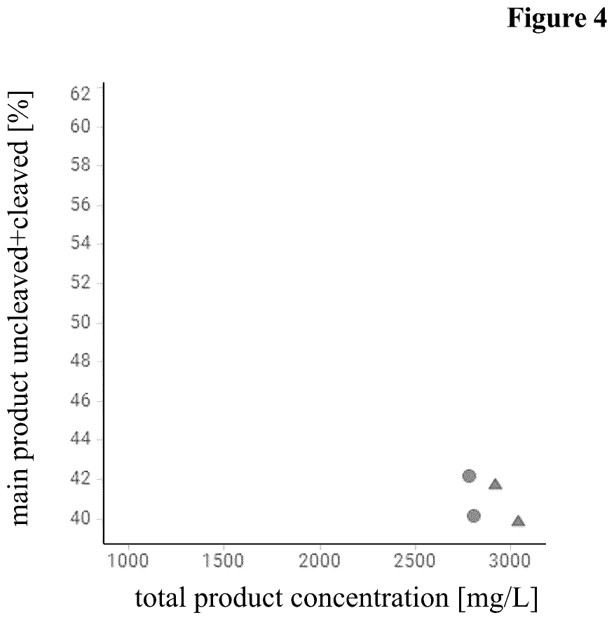

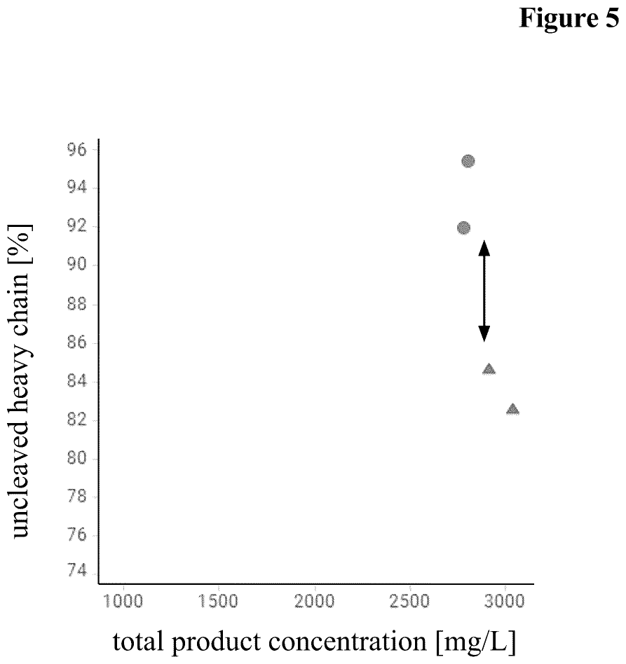

It has been found that by reducing the promoter strength of the promoter operably linked to, i.e. driving the expression of, the coding sequence of the protease inhibitor, the total titer reduction can be overcome. In more detail, by employing the medium strength SV40 promoter instead of the high strength CMV promoter the decrease in total titer is reduced whereas at the same time the inhibition of cleavage is maintained (increase of uncleaved antibody heavy chain comprising the protease cleavage site from 84% to 94%). This is shown in FIG. 4 (total titer vs. total main product) and FIG. 5 (total titer vs. not-cleaved heavy chain) (triangle: cell without inhibitor; circle: cell with co-expression of protease inhibitor driven by medium strength SV40 promoter).

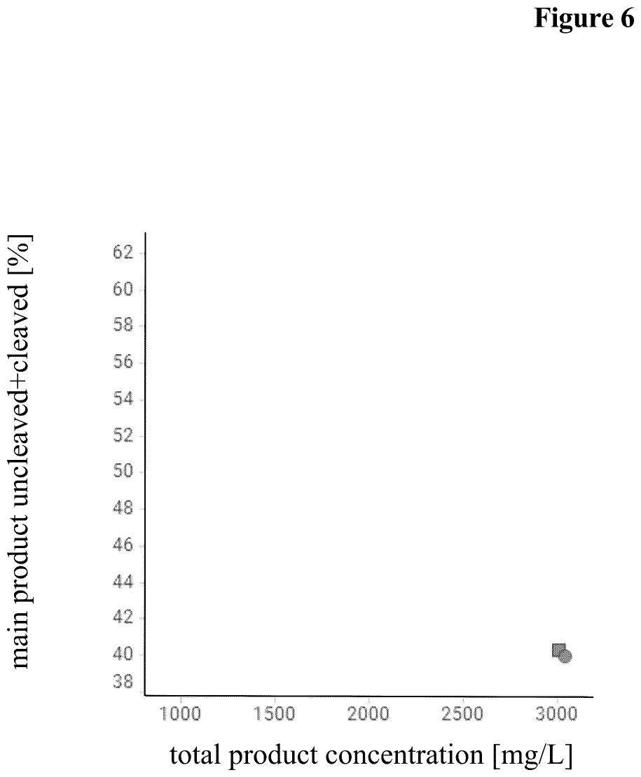

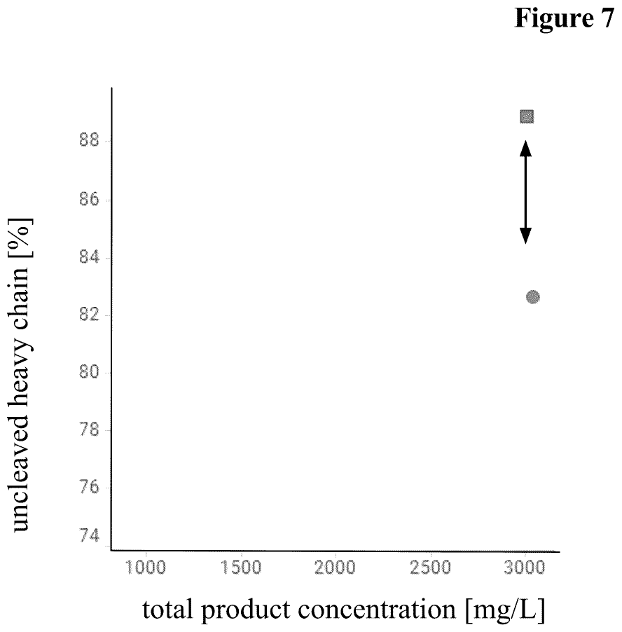

It has further unexpectedly been found that comparable results as obtained by co-expression of one protease-specific inhibitor, i.e. the extent of reduction of cleavage, could not be achieved likewise by the addition of a genus-specific protease inhibitor to the cultivation medium, although it is expected that the genus-specific protease inhibitor would have a better effect. This has been exemplarily shown by the addition of the proteinaceous serine protease inhibitor BPTI (aprotinin) to the cultivation medium (4 μM at days 3 and 10 of a 14 day fed-batch cultivation; see FIGS. 6 and 7; circle is without protease inhibitor; square is with protease inhibitor). Only an increase of 82% to 89% for the uncleaved antibody chain with the protease cleavage site (FIG. 7) could be obtained whereas the co-expression using an SV40 promoter resulted in an increase of 84% to 94% for the uncleaved antibody chain with the protease cleavage site (FIG. 5).

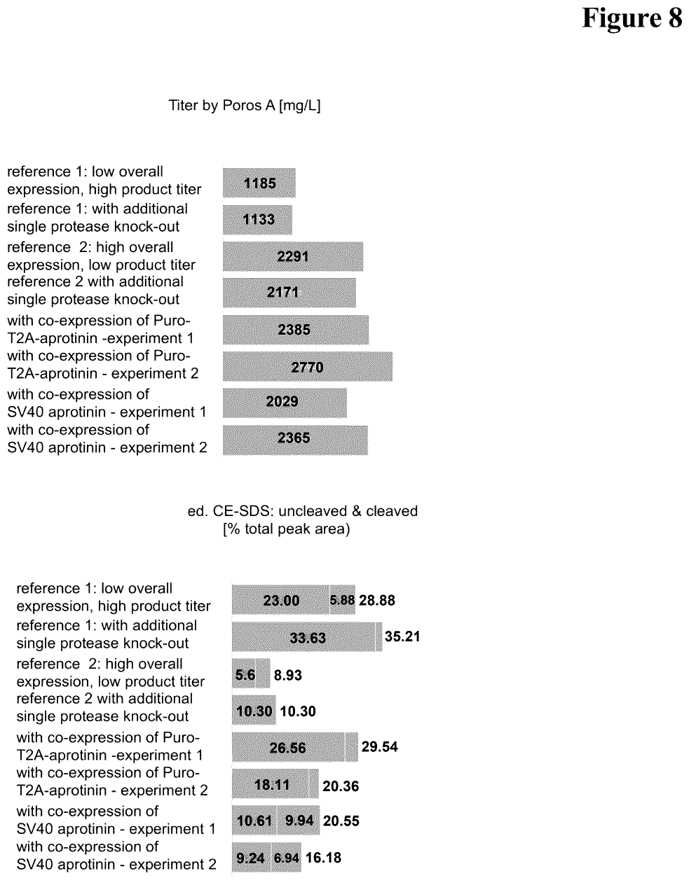

It has now unexpectedly been found that an even further improvement with respect to titer and cleavage prevention can be achieved by combining the protease inhibitor with a selection marker in a monocistronic expression cassette. This is shown in FIG. 8 and Table 2. An exemplary combination of the proteinaceous protease inhibitor BPTI (aprotinin) and the selection marker puromycin acetyltransferase linked via a T2A self-cleaving peptide sequence was used. It can be seen that on the one hand the total titer (cleaved+uncleaved) is increased (on average 2578 mg/mL (2385-2770 mg/mL) vs. on average 1862 mg/mL (1133-2365 mg/mL); in the supernatant determined by protein A chromatography) as well as the relative titer (total titer multiplied by main product (monomer) content determined by SEC) is increased (on average 1501 mg/mL (1485-1517 mg/mL) vs. on average 625 mg/mL (491-675 mg/mL) in the supernatant determined by sequential protein A and SEC chromatography). This relative titer increase is achieved by increasing the total titer (as determined by protein A chromatography) and at the same time maintaining or even improving the ratio of cleaved/uncleaved antibody heavy chain comprising the protease cleavage site (as determined by CE-SDS).

TABLE 2
Expression yield and relative titer.

| | | | rel. titer; | |
|---|---|---|---|---|
| | | | supernatant | |
| | supernatant; | main product | *main | normalize to |
| | protein A; | protein A + | product; | monocistronic |
| | [mg/L] | SEC; [%] | [mg/L] | experiment 1 |
| reference 1 (cell | 1185 | 46.16 | 547.0 | 36.8% |
| line stably | | | | |
| expressing the | | | | |
| protein of interest) | | | | |
| reference 1 + single | 1133 | 57.96 | 656.7 | 44.2% |
| protease KO | | | | |
| introduced | | | | |
| reference 2 (cell | 2291 | 21.44 | 491.2 | 33.1% |
| line stably | | | | |
| expressing the | | | | |
| protein of interest) | | | | |
| reference 2 + single | 2171 | 35.40 | 768.5 | 51.7% |
| protease KO | | | | |
| introduced | | | | |
| monocistronic | 2385 | 62.28 | 1485.4 | 100% |
| puro-T2A-BPTI | | | | |
| expression cassette | | | | |
| introduced - | | | | |
| experiment 1 | | | | |
| monocistronic | 2770 | 54.78 | 1517.4 | 102% |
| puro-T2A-BPTI | | | | |
| expression cassette | | | | |
| introduced - | | | | |
| experiment 2 | | | | |
| co-expression of | 2029 | 30.06 | 609.9 | 41.1% |
| BPTI and protein | | | | |
| of interest - | | | | |
| experiment 1 | | | | |
| co-expression of | 2365 | 28.52 | 674.5 | 45.4% |
| BPTI and protein | | | | |
| of interest - | | | | |
| experiment 2 | | | | |

Thus, in one specific realization of the teaching of the current invention the monocistronic expression cassette according to the current invention comprises a polynucleotide sequence encoding two polypeptides joined by a linker comprising a sequence capable of inducing ribosome skipping, i.e. self-cleavage.

In certain embodiments of all aspects and embodiments, the nucleic acid according to the current invention comprises a first nucleic acid encoding a selection marker and a second nucleic acid encoding a proteinaceous protease inhibitor combined by a linker sequence coding for a self-cleaving peptide sequence.

Therefore, one independent aspect of the invention is a (isolated) nucleic acid comprising in 5′- to 3′-direction in operably linked form the following elements
a) a nucleic acid encoding a selection marker,
b) a nucleic acid encoding a self-cleaving peptide sequence, and
c) a nucleic acid encoding a proteinaceous protease inhibitor.

In certain embodiments of all aspects and embodiments, the nucleic acid further comprises in operably linked form the following elements
d) a promoter upstream (5′) to the nucleic acid of a),
e) a polyadenylation signal sequence downstream (3′) to the nucleic acid of c), and
f) optionally a terminator sequence downstream (3′) to the nucleic acid of e).

In one preferred embodiment of all aspects and embodiments, the protease inhibitor inhibits plasminogen activator and/or is BPTI (aprotinin) (SEQ ID NO: 86; AQRPDFCLEPPYTGPCKARMIRYFYNAKAGLCQPFVYGGCRAKRNNFKSSEDCMRT CGGA) or the plasminogen activator inhibitor type 1 (PAI-1)-derived peptide EEIIMD (SEQ ID NO: 88).

In one preferred embodiment of all aspects and embodiments, the self-cleaving peptide sequence is the T2A self-cleaving peptide sequence or a functional variant thereof that induces ribosome skipping.

In certain embodiments of all aspects and embodiments, the T2A self-cleaving peptide sequence comprises the amino acid sequence EGRGSLLTCGDVEENPGP (SEQ ID NO: 14), which may be encoded by the nucleic acid sequence

(SEQ ID NO: 15)
GAGGGCAGAGGAAGTCTGCTAACATGCGGTGACGTCGAGGAGAATCCTG
GCCCA.

In certain embodiments of all aspects and embodiments, the linker sequence further comprises a spacer sequence before/upstream of the self-cleaving peptide sequence. In certain embodiments, the spacer sequence comprises the amino acid sequence SGRSGGG (SEQ ID NO: 03), which may be encoded by the nucleic acid sequence

(SEQ ID NO: 90)
TCCGGAAGATCTGGCGGCGGA.

In certain embodiments of all aspects and embodiments, the linker further comprises an amino acid sequence corresponding to a furin cleavage site. Furin is a protease that cleaves protein precursors before their secretion in the trans golgi. Furin cleaves at the C-terminus of its recognition sequence. Furin cleavage sequences can be added to remove amino acid residues at the C-terminus of the protein upstream of the self-cleaving peptide sequence. Different furin recognition sequences (or “furin cleavage sites”) have been developed. These include, without limitation, RXKR or RXRR, and RXXR, where X is any naturally occurring amino acid. In certain embodiments, the furin cleavage site has the recognition sequence RQKR (SEQ ID NO: 94). In certain embodiments, the furin cleavage site has the recognition sequence X1RX2X3R, wherein X1 is K or R, X2 is any naturally occurring amino acid, and X3 is K or R. The appropriate furin cleavage site for use in the present invention can be selected based on the knowledge of the invention in combination with the knowledge in the art.

In certain embodiments of all aspects and embodiments, the linker comprises a nucleic acid sequence encoding a combination of a furin cleavage site and a 2A self-cleaving peptide sequence. In certain embodiments, the linker comprises a nucleic acid sequence encoding a furin cleavage site and the F2A self-cleaving peptide sequence, a furin cleavage site and the E2A self-cleaving peptide sequence, a furin cleavage site and the P2A self-cleaving peptide sequence, or a furin cleavage site and the T2A self-cleaving peptide sequence. In certain embodiments, the linker comprises a nucleic acid sequence encoding a furin cleavage site and the T2A self-cleaving peptide sequence. The appropriate combination for use in the present invention can be selected based on the knowledge of the invention in combination with the knowledge in the art.

In certain embodiments of all aspects and embodiments, the linker may further comprise a spacer sequence between the furin cleavage site and the 2A self-cleaving peptide sequence.

Various spacer sequences are known in the art. In certain embodiments, the spacer sequence is a glycine serine (GS) spacer sequence such as (GS)ₙ, (GSGGS)ₙ (SEQ ID NO: 06) and (GGGS)ₙ (SEQ ID NO: 07), where n represents an integer of at least 1. In certain embodiments, the spacer sequence is selected from GGSG (SEQ ID NO: 08), GGSGG (SEQ ID NO: 09), GSGSG (SEQ ID NO: 10), GSGGG (SEQ ID NO: 11), GGGSG (SEQ ID NO: 12), GSSSG (SEQ ID NO: 13), GGGGS (SEQ ID NO: 99) and the like. The appropriate spacer sequence for use in the present invention can be selected based on the knowledge of the invention in combination with the knowledge in the art.

In certain embodiments of all aspects and embodiments, the nucleic acid according to the current invention comprises a nucleic acid encoding puromycin acetyltransferase and a nucleic acid encoding BPTI that are separated by a furin cleavage site-(G4S)₂-T2A self-cleaving peptide sequence (F-G4S2-T2A linker). The F-G4S2-T2A linker has the amino acid sequence RAKRGGGGSGGGGSEGRGSLLTCGDVEENPGP (SEQ ID NO: 106) and may be encoded by the nucleic acid sequence

(SEQ ID NO: 107)
AGAGCCAAGCGCGGCGGCGGCGGCAGCGGCGGCGGCGGCAGCGAGGGCA
GAGGAAGTCTGCTAACATGCGGTGACGTCGAGGAGAATCCTGGCCCA.

In certain alternative embodiments of the above, the self-cleaving peptide sequence is the F2A self-cleaving peptide sequence. In certain embodiments, the F2A self-cleaving peptide sequence comprises the amino acid sequence VKQTLNFDLLKLAGDVESNPGP (SEQ ID NO: 44), which may be encoded by the nucleic acid sequence (SEQ ID NO: 45)

GTGAAACAGACTTTGAATTTTGACCTTCTCAAGTTGGCGGGAGACGTGG
AGTCCAACCCAGGGCCG.

A “functional fragment” of a polypeptide, e.g. an antibody, self-cleaving peptide sequence, enzyme, selection marker, or a nucleic acid, is a polypeptide or a nucleic acid whose sequence is not identical to the respective full-length polypeptide or nucleic acid, but retains the same function as the full-length polypeptide or nucleic acid. Thus, the term “functional fragment” encompasses variants of the full-length polypeptide or nucleic acid that possess more or fewer residues as the corresponding full-length molecule, i.e. is shorter or longer, and/or comprises one or more amino acid or nucleotide substitutions. Methods for determining the function of a nucleic acid (e.g., coding function, ability to hybridize to another nucleic acid) as well as of polypeptides are well-known in the art. See, e.g., Ausubel et al, supra; Fields et al. (1989) Nature 340:245-246; U.S. Pat. No. 5,585,245 and WO 98/44350.

### The Cell and the Method According to the Current Invention

The invention is exemplified with a CHO cell and using targeted integration. This is presented solely to exemplify the invention but shall not be construed in any way as limitation. Any other eukaryotic or mammalian cell and any other transfection/integration method can be used. The true scope of the invention is set forth in the claims.

For example, in targeted integration (TI), site-specific recombination is employed for the introduction of an exogenous nucleic acid into a specific locus in the genome of a mammalian TI host cell. This can be used to generate recombinant cells according to the invention. TI is an enzymatic process wherein a sequence at the site of integration in the genome is exchanged for the exogenous nucleic acid. One system used to effect such nucleic acid exchanges is the Cre-lox system. The enzyme catalyzing the exchange is the Cre recombinase. The sequence to be exchanged is defined by the position of at least two lox(P)-sites in the genome as well as in the exogenous nucleic acid. These lox(P)-sites are recognized by the Cre recombinase. Nothing more is required, i.e. no ATP etc.

A suitable mammalian TI host cell used in this exemplification of the method according to the invention for generating a recombinant cell according to the invention is a CHO cell harboring a landing site integrated at a single site within a locus of its genome wherein the landing site comprises three heterospecific loxP sites for Cre recombinase mediated DNA recombination.

In more detail, the heterospecific loxP sites are L3, LoxFas and 2L (see e.g. Lanza et al., Biotechnol. J. 7 (2012) 898-908; Wong et al., Nucleic Acids Res. 33 (2005) e147), whereby L3 and 2L flank the landing site at the 5′-end and 3′-end, respectively, and LoxFas is located between the L3 and 2L sites. The landing site further contains a bicistronic unit linking the expression of a selection marker via an IRES to the expression of the fluorescent GFP protein allowing to stabilize the landing site by positive selection as well as to select for the absence of the site after transfection and Cre-recombination (negative selection). Green fluorescence protein (GFP) serves for monitoring the recombinase mediated cassette exchange (RMCE) reaction.

Such a configuration of the landing site as outlined in the previous paragraph allows for the simultaneous integration of two vectors, e.g. of a so called front vector harboring an L3 and a LoxFas site and a back vector harboring a LoxFas and an 2L site. The functional elements of a selection marker different from that present in the landing site and also different from that in the nucleic acid according to the invention have been distributed between both vectors: promoter and start codon were located on the front vector whereas coding region and polyadenylation signal sequence were located on the back vector. Only correct recombinase-mediated integration of said nucleic acids from both vectors induces resistance against the respective selection agent.

Thus, two vectors have been designed: A first vector, the front vector, and a second vector, the back vector. Both vectors comprise different expression cassettes. The number of expression cassettes is generally not limited but generally is independently between 1 and 4 expression cassettes per vector. One of the expression cassettes, e.g. in the back vector, comprises the nucleic acid according to the invention.

In the current example for the expression of an N-terminal Fab-domain inserted 2+1 bispecific antibody with additional functional group connected by a peptidic linker a front vector comprising in the following order an L3-site, an expression cassette of the first heavy chain, a first expression cassette for the first light chain, a second expression cassette for the first light chain, and the promoter, the start codon of a selection marker different from puromycin acetyltransferase and a LoxFas site as well as a back vector comprising a LoxFas site, the coding region and polyadenylation signal sequence of the selection marker, an expression cassette encoding the second heavy chain, an expression cassette encoding the second light chain, an expression cassette comprising the nucleic acid according to the invention and a 2L-site were used. These have been integrated using double RMCE into the CHO-K1 TI host cell, cultivated and the heterologous antibody product isolated from the supernatant. The respective data is presented in the preceding section.

Thus, in certain embodiments of all aspects and embodiments, the nucleic acid according to the invention and the one or more nucleic acids encoding the heterologous polypeptide have been integrated into a mammalian TI host cell by double recombinase mediated cassette exchange (RMCE). Thereby a recombinant mammalian cell according to the invention, such as a recombinant CHO cell, is obtained, in which the expression cassettes have been integrated into the genome at a single locus.

In certain embodiments of all aspects and embodiments, the integrated landing site comprises at least one selection marker. In certain embodiments, the integrated landing site comprises a first, a second and a third recombination recognition sequence (RRS), and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments of all aspects and embodiments, the first, second and third RRS are the L3 (SEQ ID NO: 96), LoxFas (SEQ ID NO: 98) and 2L (SEQ ID NO: 97) sites.

In certain embodiments of all aspects and embodiments, the CHO cell is a CHO-K1 cell.

Thus, one aspect of the invention is a method for preparing a recombinant cell expressing a heterologous polypeptide comprising:
a) providing a targeted integration host cell comprising an exogenous nucleotide sequence integrated at a site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a first vector comprising two recombination recognition sequences that are matching the first and the third recombination recognition sequence on the integrated exogenous nucleotide sequence, wherein the two recombination recognition sequences are flanking two to four expression cassettes (exogenous nucleotide sequences) and at least (a part of) one second selection marker, and a second vector comprising two recombination recognition sequences that are matching the second and the third recombination recognition sequence on the integrated exogenous nucleotide sequence, wherein the two recombination recognition sequences are flanking two to four (further) expression cassettes (exogenous nucleotide sequences), wherein at least one expression cassette comprises the nucleic acid according to the invention;
c) introducing i) either simultaneously with the first and second vectors of b); or ii) sequentially thereafter one or more recombinases,
wherein the one or more recombinases recognize the recombination recognition sequences of the first and second vectors; (and optionally wherein the one or more recombinases perform two recombinase mediated cassette exchanges;)
and
d) selecting for recombinant host cells expressing the second selection marker and secreting a bispecific antibody,
thereby preparing a recombinant cell expressing a heterologous polypeptide.

In certain embodiments of all aspects and embodiments, the first or/and the second vector comprises an expression cassette comprising the nucleic acid according to the invention.

In certain embodiments of all aspects and embodiments, the heterologous polypeptide is a multispecific antibody. In one preferred embodiment, the heterologous polypeptide is a bispecific antibody.

In certain embodiments of all aspects and embodiments, each of the first and the second vector comprises at least one exogenous nucleotide sequence encoding an antibody light chain and at least one exogenous nucleotide sequence encoding an antibody heavy chain.

In certain embodiments of all aspects and embodiments, the first or/and the second vector comprises one exogenous nucleotide sequence encoding an antibody light chain and one exogenous nucleotide sequence encoding an antibody heavy chain.

In certain embodiments of all aspects and embodiments, the first or/and the second vector comprises one exogenous nucleotide sequence encoding an antibody light chain and one exogenous nucleotide sequence encoding an antibody heavy chain, wherein the exogenous nucleotide sequence encoding the antibody heavy chain is located upstream (5′) to the exogenous nucleotide sequence encoding the antibody light chain.

In certain embodiments of all aspects and embodiments, the first or/and the second vector comprises one exogenous nucleotide sequence encoding an antibody light chain and one exogenous nucleotide sequence encoding an antibody heavy chain, wherein the antibody light chain and the antibody heavy chain have a domain crossover.

In certain embodiments of all aspects and embodiments, the first vector comprises one exogenous nucleotide sequence encoding an antibody light chain and one exogenous nucleotide sequence encoding an antibody heavy chain, wherein the antibody light chain and the antibody heavy chain have a domain crossover.

In one preferred embodiment of all aspects and embodiments, the first vector comprises one exogenous nucleotide sequence encoding an antibody light chain and one exogenous nucleotide sequence encoding an antibody heavy chain, wherein the antibody light chain and the antibody heavy chain have a domain crossover and the exogenous nucleotide sequence encoding the antibody light chain with domain crossover is located upstream (5′) to the exogenous nucleotide sequence encoding the antibody heavy chain with domain crossover.

In one preferred embodiment of all aspects and embodiments, the first vector comprises one exogenous nucleotide sequence encoding an antibody light chain and one exogenous nucleotide sequence encoding an antibody heavy chain, wherein the antibody light chain and the antibody heavy chain have a domain crossover and the exogenous nucleotide sequence encoding the antibody heavy chain with domain crossover is located upstream (5′) to the exogenous nucleotide sequence encoding the antibody light chain with domain crossover.

In one embodiment of all aspects and embodiments, the first vector comprises a promoter sequence operably linked to the codon ATG, whereby the promoter sequence is flanked upstream by (i.e. positioned downstream to) the (two) exogenous nucleotide sequences and the ATG codon is flanked downstream by (i.e. positioned upstream to) a recombination recognition sequence; and the second vector comprises a selection marker lacking an ATG transcription start codon flanked upstream by a recombination recognition sequence and downstream by the (two) exogenous nucleotide sequences.

One further aspect of the invention is a method of producing a heterologous polypeptide comprising:
a) providing a recombinant cell according to the invention;
b) culturing the recombinant cell of a) and recovering the heterologous polypeptide from the cell or the cultivation medium;
c) optionally purifying the heterologous polypeptide by one or more chromatography steps;
and thereby producing a heterologous polypeptide.

### Cells

Any mammalian cell can be used to generate recombinant cells according to the invention, which can be used in the method according to the current invention. That is, independent from the integration method, i.e. for random integration (RI) as well as TI, any mammalian cell can be used.

Examples of useful mammalian cells are human amniocyte cells (e.g. CAP-T cells as described in Woelfel, J. et al., BMC Proc. 5 (2011) P133); monkey kidney CV1 line transformed by SV40 (COS-7); human embryonic kidney line (HEK293 or HEK293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian cells include Chinese hamster ovary (CHO) cells, including DHFR-CHO cells (Urlaub, G. et al., Proc. Natl. Acad. Sci. USA 77 (1980) 4216-4220); and myeloma cells such as Y0, NS0 and Sp2/0. For a review of certain mammalian cells suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

The term “recombinant cell” as used herein denotes a mammalian cell comprising an exogenous nucleic acid. Such recombinant mammalian cells are cells into which at least the nucleic acid according to the invention has been introduced, including the progeny of such cells. In certain embodiments, the recombinant cell is a mammalian cell comprising the nucleic acid according to the invention and one or more further nucleic acids encoding a heterologous polypeptide. Thus, the term “recombinant cell comprising a nucleic acid encoding a heterologous polypeptide” denotes recombinant mammalian cells comprising one or more exogenous nucleic acids integrated in the genome of the mammalian cell and capable of expressing the heterologous polypeptide as well as the nucleic acid according to the invention. In certain embodiments, the recombinant cell is a mammalian cell comprising one or more exogenous nucleic acids integrated at a single site within a locus of the genome of the cell. In one preferred embodiment, the recombinant cell is a mammalian cell comprising the nucleic acid according to the invention and one or more further exogenous nucleic acids integrated at a single site within a locus of the genome of the cell, wherein the integrated nucleic acid comprises a first, a second and a third recombination recognition sequence located between the first and the second recombination recognition sequence, and all the recombination recognition sequences are different.

A “recombinant cell” further encompasses a cell after genetic modification, such as, e.g., a cell comprising the nucleic acid according to the invention and expressing a heterologous polypeptide of interest and which can be used for the recombinant production of said heterologous polypeptide of interest at any scale. For example, “a recombinant cell” denotes a cell wherein the nucleic acid according to the invention and one or more nucleic acids encoding a heterologous polypeptide of interest have been stably introduced into the genome. For example, “a recombinant mammalian cell comprising the nucleic acid according to the invention and one or more nucleic acids encoding a heterologous polypeptide” may be a mammalian cell that has been subjected to recombinase mediated cassette exchange (RMCE), whereby the nucleic acid according to the invention and the coding sequences for a polypeptide of interest have been stably introduced into the genome of a mammalian cell.

A “recombinant cell” further includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated cell” denotes a cell which has been separated from a component of its natural environment.

An “isolated nucleic acid” denotes a nucleic acid molecule that has been separated from a component of its natural environment.

In certain embodiments of all aspects and embodiments, the mammalian cell is, e.g., a Chinese Hamster Ovary (CHO) cell (e.g. CHO K1, CHO DG44, etc.), a Human Embryonic Kidney (HEK) cell, a lymphoid cell (e.g., Y0, NS0, Sp2/0 cell), or a human amniocyte cells (e.g. CAP-T, etc.). In one preferred embodiment of all aspects and embodiments, the cell is a CHO cell.

With respect to TI, any known or future mammalian cell suitable for TI comprising a landing site as described herein integrated at a single site within a locus of the genome can be used in the current invention. Such a cell can be denoted as a mammalian TI host cell. In certain embodiments, the mammalian TI host cell is a hamster cell, a human cell, a rat cell, or a mouse cell comprising a landing site as described herein. In one preferred embodiment, the mammalian TI host cell is a CHO cell. In certain embodiments, the mammalian TI host cell is a Chinese hamster ovary (CHO) cell, a CHO K1 cell, a CHO K1SV cell, a CHO DG44 cell, a CHO DUKXB-11 cell, a CHO K1S cell, or a CHO K1M cell comprising a landing site as described herein integrated at a single site within a locus of the genome.

### Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

The term “antibody” herein is used in the broadest sense and encompasses various antibody structures, including but not limited to full length antibodies, monoclonal antibodies, multispecific antibodies (e.g., bispecific antibodies), and antibody-antibody fragment-fusions as well as combinations thereof as long as these comprises one or more amino acid sequences that is/are cleaved during recombinant production by a protease endogeneous to the mammalian cell used for expression in the absence of the nucleic acid according to the invention or a functional variant thereof.

The term “full length antibody” denotes an antibody having a structure substantially similar to that of a native antibody. A full length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full-length antibody.

The term “antibody binding site” denotes a pair of a heavy chain variable domain and a light chain variable domain. To ensure proper binding to the antigen these variable domains are cognate variable domains, i.e. belong together. An antibody binding site comprises at least three HVRs (e.g. in case of a VHH) or three-six HVRs (e.g. in case of a naturally occurring, i.e. conventional, antibody with a VH/VL pair). Generally, the amino acid residues of an antibody that are responsible for antigen binding are forming the binding site. These residues are normally contained in a pair of an antibody heavy chain variable domain and a corresponding antibody light chain variable domain. The antigen-binding site of an antibody comprises amino acid residues from the “hypervariable regions” or “HVRs”. “Framework” or “FR” regions are those variable domain regions other than the hypervariable region residues as defined herein. Therefore, the light and heavy chain variable domains of an antibody comprise from N- to C-terminus the regions FR1, HVR1, FR2, HVR2, FR3, HVR3 and FR4. Especially, the HVR3 region of the heavy chain variable domain is the region which contributes most to antigen binding and defines the binding specificity of an antibody. A “functional binding site” is capable of binding to its target. The term “binding to” denotes the binding of a binding site to its target in an in vitro assay, in certain embodiments, in a binding assay. Such binding assay can be any assay as long as the binding event can be detected. “Binding” can be determined using, for example, an ELISA assay.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the heavy chain variable domain VH (H1, H2, H3), and three in the light chain variable domain VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 6, F, 7, and p, respectively.

The term “heavy chain constant region” denotes the region of an immunoglobulin heavy chain that contains the constant domains, i.e. the CH1 domain, the hinge region, the CH2 domain and the CH3 domain. In one embodiment, a human IgG constant region extends from Alal 18 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the CH2 domain and the CH3 domain. In one embodiment, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The term “valent” as used within the current application denotes the presence of a specified number of binding sites in an antibody. As such, the terms “bivalent”, “tetravalent”, and “hexavalent” denote the presence of two binding sites, four binding sites, and six binding sites, respectively, in an antibody.

A “monospecific antibody” denotes an antibody that has a single binding specificity, i.e. specifically binds to one antigen. Monospecific antibodies can be prepared as full-length antibodies or antibody fragments (e.g. F(ab′)2) or combinations thereof (e.g. full length antibody plus additional scFv or Fab fragments). A monospecific antibody does not need to be monovalent, i.e. a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g. Fab bispecific antibodies) or combinations thereof (antibody-antibody fragment-fusions, e.g. a full-length antibody conjugated to an additional scFv or Fab fragments). A multispecific antibody is at least bivalent, i.e. comprises two antigen binding sites. In addition, a multispecific antibody is at least bispecific. Thus, a bivalent, bispecific antibody is the simplest form of a multispecific antibody. Engineered antibodies with two, three or more (e.g. four) functional antigen binding sites have also been reported (see, e.g., US 2002/0004587).

In certain embodiments of all aspects and embodiments, the cell produces a multispecific antibody as heterologous polypeptide. In certain embodiments, one of the binding specificities of the multispecific antibody is for a first antigen and the other is for a different second antigen. In certain embodiments, the multispecific antibody binds to two different epitopes of the same antigen. In certain embodiments, the second epitope on the same antigen is a non-overlapping epitope. In certain embodiments, the antibody is a bispecific antibody. In one preferred embodiment, the bispecific antibody is a trivalent, bispecific antibody or a bivalent, bispecific antibody.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553); using the common light chain technology for circumventing the light chain mis-pairing problem (see, e.g., WO 98/50431); using specific technology for making bispecific antibody fragments (see, e.g., Holliger, P., et al., Proc. Natl. Acad. Sci. USA 90 (1993) 6444-6448); and preparing trispecific antibodies as described, e.g., in Tutt, A., et al., J. Immunol. 147 (1991) 60-69).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see, e.g., WO 2009/080252 and WO 2015/150447), the CH1/CL domains (see, e.g., WO 2009/080253) or the complete Fab arms (see e.g., WO 2009/080251, WO 2016/016299, also see Schaefer et al., Proc. Natl. Acad. Sci. USA 108 (2011) 1187-1191, and Klein at al., MAbs 8 (2016) 1010-1020).

In one preferred embodiment of all aspects and embodiments, the multispecific antibody comprises a Fab fragment, in which either the variable regions or the constant regions of the heavy and light chain are exchanged, i.e. wherein in one chain a heavy chain VH variable domain is either directly of via a peptidic linker conjugated to a light chain CL constant domain, and in the respective other chain a light chain VL variable domain is either directly of via a peptidic linker conjugated to a heavy chain CH1 constant domain.

Thus, a domain exchanged Fab fragment comprises a polypeptide chain composed of the light chain variable region (VL) and the heavy chain constant region 1 (CH1), and a polypeptide chain composed of the heavy chain variable region (VH) and the light chain constant region (CL).

Asymmetrical Fab arms can also be engineered by introducing charged or non-charged amino acid mutations into domain interfaces to direct correct Fab pairing. See e.g., WO 2016/172485.

The antibody or fragment can also be a multispecific antibody as described in WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Various further molecular formats for multispecific antibodies are known in the art and are included herein (see e.g., Spiess et al., Mol. Immunol. 67 (2015) 95-106).

Bispecific antibodies are generally antibody molecules that specifically bind to two different, non-overlapping epitopes on the same antigen or to two epitopes on different antigens.

In certain embodiments of all aspects and embodiments, the bispecific antibody is selected form the group of bispecific antibodies consisting of
a domain exchanged 1+1 bispecific antibody (CrossMab);
   such an antibody is a bispecific, full-length IgG antibody comprising a pair of a first light chain and a first heavy chain comprising a first Fab fragment and a pair of a second light chain and a second heavy chain comprising a second Fab fragment,
   wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains and the VH and VL domains are replaced by each other (i.e. the light chain of the first Fab fragment comprises a VH and a CH1 domain and the heavy chain of the first Fab fragment comprises a VL and a CL domain);
   wherein the second Fab fragment comprises a light chain comprising a VL and a CL domain, and a heavy chain comprising a VH and a CH1 domain;
   wherein the first heavy chain and the second heavy chain both comprise a CH3 domain, wherein both CH3 domains are engineered in a complementary manner by respective amino acid substitutions, in order to support heterodimerization of the first heavy chain and the second heavy chain, (in one preferred embodiment, one CH3 domain comprises the knob-mutation and the respective other CH3 domain comprises the hole-mutations;
a C-terminal Fab domain fused 2+1 bispecific antibody (BS);
   such an antibody is a bispecific, full length IgG antibody comprising
   a) one full length antibody comprising two pairs each of a full length antibody light chain and a full length antibody heavy chain, wherein the binding sites formed by each of the pairs of the full length heavy chain and the full length light chain specifically bind to a first antigen, and
   b) one additional Fab fragment, wherein the additional Fab fragment is fused to the C-terminus of one heavy chain of the full length antibody, wherein the binding site of the additional Fab fragment specifically binds to a second antigen,
   wherein the additional Fab fragment specifically binding to the second antigen comprises a domain crossover such that a) the light chain variable domain (VL) and the heavy chain variable domain (VH) are replaced by each other, or b) the light chain constant domain (CL) and the heavy chain constant domain (CH1) are replaced by each other;
a bispecific, one-armed single chain antibody (OaMab);
   such an antibody is a bispecific, one-armed single chain antibody comprising a first binding site that specifically binds to a first epitope or antigen and a second binding site that specifically binds to a second epitope or antigen, whereby the individual chains are as follows
      a light chain (comprising variable light chain domain and light chain constant domain);
      a combined light/heavy chain (comprising in N- to C-terminal order a variable light chain domain, a light chain constant domain, peptidic linker, variable heavy chain domain, a CH1 domain, a hinge region, a CH2 domain and a CH3 with knob- or hole-mutation)
      a heavy chain (comprising in N- to C-terminal order a variable heavy chain domain, a CH1 domain, a hinge region, a CH2 domain, a CH3 domain with hole- or knob-mutation));
a bispecific, two-armed single chain antibody;
   such an antibody is a bispecific, two-armed single chain antibody comprising a first binding site that specifically binds to a first epitope or antigen and a second binding site that specifically binds to a second epitope or antigen, whereby the individual chains are as follows
      a combined light/heavy chain 1 (comprising in N- to C-terminal order a variable light chain domain 1, a light chain constant domain, a peptidic linker, a variable heavy chain domain 1, a CH1 domain, a hinge region, a CH2 domain, a CH3 domain with knob- or hole-mutation);
      combined light/heavy chain 2 (comprising in N- to C-terminal order a variable light chain domain 2, a light chain constant domain, a peptidic linker, a variable heavy chain domain 2, a CH1 domain, a hinge region, a CH2 domain, a CH3 domain with hole- or knob-mutation);
N-terminal Fab-domain inserted 2+1 bispecific antibody (TCB);
   such an antibody is a bispecific, full-length antibody with additional heavy chain N-terminal binding site with domain exchange comprising
      a first and a second Fab fragment, wherein each binding site of the first and the second Fab fragment specifically bind to a first antigen,
      a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
      an Fc-region comprising a first Fc-region polypeptide and a second Fc-region polypeptide,
   wherein the first and the second Fab fragment each comprise a heavy chain fragment and a full-length light chain,
   wherein the C-terminus of the heavy chain fragment of the first Fab fragment is fused to the N-terminus of the first Fc-region polypeptide,
   wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide;
an antibody-multimer-fusion;
   such an antibody is a fusion polypeptide comprising
   (a) an antibody heavy chain and an antibody light chain, and
   (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
   wherein
   (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
   wherein
   the variable domains of the antibody heavy chain and the antibody light chain form a binding site specifically binding to an antigen).

The CH3 domains in the heavy chains of an antibody can be altered by the “knob-into-holes” technology, which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method, the interaction surfaces of the two CH3 domains are altered to increase the heterodimerization of these two CH3 domains and thereby of the polypeptide comprising them. Each of the two CH3 domains (of the two heavy chains) can be the “knob”, while the respective other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

The mutation T366W in the CH3 domain (of an antibody heavy chain) is denoted as “knob-mutation” and the mutations T366S, L368A, Y407V in the CH3 domain (of an antibody heavy chain) are denoted as “hole-mutations” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations”) and by introducing a Y349C mutation into the CH3 domain of the heavy chain with the “hole-mutations” (denotes as “hole-cys-mutations”) (numbering according to Kabat EU index).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci. USA 108 (2011) 11187-11192. Such antibodies are generally termed CrossMab.

Multispecific antibodies also comprise, in certain embodiments, at least one Fab fragment including either a domain crossover of the CH1 and the CL domains as mentioned under item (i) above, or a domain crossover of the VH and the VL domains as mentioned under item (ii) above, or a domain crossover of the VH-CH1 and the VL-VL domains as mentioned under item (iii) above. In case of multispecific antibodies with domain crossover, the Fabs specifically binding to the same antigen(s) are constructed, in certain embodiments, to be of the same domain sequence. Hence, in case more than one Fab with a domain crossover is contained in the multispecific antibody, said Fab(s) specifically bind to the same antigen.

The term “recombinant antibody”, as used herein, denotes all antibodies (chimeric, humanized and human) that are prepared, expressed, created or isolated by recombinant means, such as recombinant cells. This includes antibodies isolated from recombinant cells such as NS0, HEK, BHK, amniocyte or CHO cells.

As used herein, the term “antibody fragment” refers to a molecule other than an intact antibody that comprises a portion of an intact antibody that binds the antigen to which the intact antibody binds, i.e. it is a functional fragment. Examples of antibody fragments include but are not limited to Fv; Fab; Fab′; Fab′-SH; F(ab′)₂; bispecific Fab; diabodies; linear antibodies; single-chain antibody molecules (e.g., scFv or scFab).

### Recombinant Methods

Antibodies may be produced using recombinant methods and compositions, e.g., as described in U.S. Pat. No. 4,816,567. For these methods, one or more isolated nucleic acid(s) encoding the antibody are provided.

In one aspect of the invention, a method of recombinantly producing an antibody comprising one or more amino acid sequences cleavable and cleaved during recombinant production in the absence of the nucleic acid according to the current invention by an endogenous protease of the producing cell is provided, wherein the method comprises culturing a recombinant cell according to the invention comprising a nucleic acid according to the invention and one or more nucleic acid(s) encoding the antibody, under conditions suitable for expression of the antibody, and optionally recovering the antibody from the recombinant cell (and/or the cell culture medium) and further optionally purifying the antibody by one or more chromatography steps.

For recombinant production of an antibody, nucleic acids encoding the antibody are generated/designed/synthesized and inserted into one or more vectors for further cloning and/or expression in a cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Generally, for the recombinant large-scale production of a polypeptide of interest, such as e.g. a therapeutic antibody, a recombinant cell stably expressing and secreting said polypeptide is required. The overall process used for generating such a recombinant cell is termed “cell line development”. In the first step of the cell line development process, a suitable mammalian cell, such as, e.g., in certain embodiments, a CHO cell, is transfected with one or more nucleic acids including the nucleic acid according to the invention and those suitable for expression of said polypeptide of interest. In a second step, recombinant cells stably expressing the proteinaceous protease inhibitor and the polypeptide of interest are selected, e.g. based on the co-expression of a selection marker, which had been co-transfected with the nucleic acids.

The nucleotide sequence of a nucleic acid encoding a polypeptide, i.e. the coding sequence, is denoted as a structural gene. Such a structural gene is pure coding information. Thus, additional regulatory elements are required for expression thereof. Therefore, normally a structural gene is integrated in a so-called expression cassette. The minimal regulatory elements needed for an expression cassette to be functional in a mammalian cell are a promoter functional in said mammalian cell, which is located upstream, i.e. 5′, to the structural gene, and a polyadenylation signal sequence functional in said mammalian cell, which is located downstream, i.e. 3′, to the structural gene. The promoter, the structural gene and the polyadenylation signal sequence are arranged in an operably linked form.

In case the polypeptide of interest is a heteromultimeric polypeptide that is composed of different polypeptides, such as e.g. an antibody or a complex antibody format, not only a single expression cassette is required but a multitude of expression cassettes differing in the respectively contained structural gene, i.e. at least one expression cassette for each of the different polypeptides (chains) of the heteromultimeric polypeptide (heteromultimeric antibody). For example, a full-length antibody is a heteromultimeric polypeptide comprising two copies of a light chain as well as two copies of a heavy chain. Thus, a full-length antibody is composed of two different polypeptides. Therefore, two expression cassettes are required for the expression of a full-length antibody, one for the light chain and one for the heavy chain. If, for example, the full-length antibody is a bispecific antibody, i.e. the antibody comprises two different binding sites specifically binding to two different antigens/epitopes on the same antigen, the two light chains as well as the two heavy chains are also different from each other. Thus, such a bispecific, full-length antibody is composed of four different polypeptides and therefore, four expression cassettes are required.

The expression cassette(s) for the polypeptide of interest is(are) in turn integrated into one or more so called “expression vector(s)” for direct expression or “integration vectors” for targeted integration. A “vector” is a nucleic acid providing all required elements for the amplification of said vector in bacterial cells as well as the expression of the comprised structural gene(s) in a mammalian cell. Typically, an expression vector comprises a prokaryotic plasmid propagation unit, e.g. for E. coli, comprising an origin of replication, and a prokaryotic selection marker, as well as a eukaryotic selection marker, and the expression cassettes required for the expression of the structural gene(s) of interest. An “expression vector” or “integration vector” is a transport vehicle for the introduction of expression cassettes into a mammalian cell to generate recombinant polypeptide-expressing cells.

As outlined in the previous paragraphs, the more complex the polypeptide to be expressed is, the higher also the number of required different expression cassettes will be. Inherently with the number of expression cassettes also the size of the nucleic acid to be integrated into the genome of the cell increases. Concomitantly also the size of the expression vector increases. However, there is a practical upper limit to the size of a vector in the range of about 15 kbps above which handling and processing efficiency profoundly drops. This issue can be addressed by using two or more expression vectors. Thereby the expression cassettes can be split between different expression vectors each comprising only some of the expression cassettes resulting in a size reduction.

Cell line development (CLD) for the generation of recombinant cell expressing a heterologous polypeptide, such as e.g. a multispecific antibody, employs either random integration (RI) or targeted integration (TI) of the nucleic acid(s) comprising the respective expression cassettes required for the expression and production of the heterologous polypeptide of interest.

Using RI, in general, several vectors or fragments thereof integrate into the cell's genome at the same or different loci.

Using TI, in general, a single copy of the transgene comprising the different expression cassettes is integrated at a predetermined “hot-spot” in the cell's genome.

Suitable cells for the generation of recombinant cells for expression of an (glycosylated) antibody are generally derived from multicellular organisms such as, e.g., vertebrates.

### Targeted Integration

One method for the generation of a recombinant mammalian cell according to the current invention to be used in the method according to the current invention is targeted integration for the introduction of the respective nucleic acids.

In certain embodiments of all aspects and embodiments, the nucleic acid according to the invention and the one or more nucleic acids encoding the heterologous polypeptide have been integrated into the mammalian TI host cell by single or double recombinase mediated cassette exchange (RMCE). Thereby a recombinant mammalian cell, such as a recombinant CHO cell, is obtained, in which the expression cassettes have been integrated into the genome at a single locus.

The Cre-LoxP site-specific recombination system has been widely used in many biological experimental systems. Cre recombinase is a 38-kDa site-specific DNA recombinase that recognizes 34 bp LoxP sequences. Cre recombinase is derived from bacteriophage P1 and belongs to the tyrosine family site-specific recombinases. Cre recombinase can mediate both intra- and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre recombinase-mediated recombination will excise DNA sequences located between the two LoxP sequences as a covalently closed circle. If two LoxP sequences are placed in an inverted position on the same nucleotide sequence, Cre recombinase-mediated recombination will invert the orientation of the DNA sequences located between the two sequences. If two LoxP sequences are on two different DNA molecules and if one DNA molecule is circular, Cre recombinase-mediated recombination will result in integration of the circular DNA sequence.

A “recombination recognition sequence” (RRS) is a nucleotide sequence recognized by a recombinase and is necessary and sufficient for recombinase-mediated recombination events.

A RRS can be used to define the position where a recombination event will occur in a nucleotide sequence.

The term “matching RRSs” indicates that a recombination occurs between two RRSs. In certain embodiments, the two matching RRSs are the same.

In certain embodiments of all aspects and embodiments, a RRS can be recognized by a Cre recombinase.

In certain embodiments of all aspects and embodiments, a RRS can be recognized by a FLP recombinase.

In certain embodiments of all aspects and embodiments, a RRS can be recognized by a Bxb1 integrase.

In certain embodiments of all aspects and embodiments, a RRS can be recognized by a φC31 integrase.

In certain embodiments of all aspects and embodiments, both RRSs are wild-type LoxP sequences. In certain embodiments, both RRSs are mutant LoxP sequences.

In certain embodiments of all aspects and embodiments, both RRSs are wild-type FRT sequences.

In certain embodiments of all aspects and embodiments, both RRSs are mutant FRT sequences.

In certain embodiments of all aspects and embodiments, the two matching RRSs are different sequences but can be recognized by the same recombinase.

In certain embodiments of all aspects and embodiments, the first matching RRS is a Bxb1 attP sequence and the second matching RRS is a Bxb1 attB sequence.

In certain embodiments of all aspects and embodiments, the first matching RRS is a φC31 attB sequence and the second matching RRS is a φC31 attB sequence.

A “two-plasmid RMCE” strategy or “double RMCE” is employed in the method according to the current invention when using a two-vector combination. For example, but not by way of limitation, an integrated landing site could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

The two-plasmid RMCE strategy involves using three RRS sites to carry out two independent RMCEs simultaneously. Therefore, a landing site in the mammalian TI host cell using the two-plasmid RMCE strategy includes a third RRS site (RRS3) that has no cross activity with either the first RRS site (RRS1) or the second RRS site (RRS2). The two plasmids to be targeted require the same flanking RRS sites for efficient targeting, one plasmid (front) flanked by RRS1 and RRS3 and the other (back) by RRS3 and RRS2. In addition, two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in frame translation for the fusion protein, i.e. operable linkage. Only when both plasmids are correctly inserted, the full expression cassette of the selection marker will be assembled and, thus, render cells resistant to the respective selection agent.

Two-plasmid RMCE involves double recombination crossover events, catalyzed by a recombinase, between the two heterospecific RRSs within the target genomic locus and the donor DNA molecule. Two-plasmid RMCE is designed to introduce a copy of the DNA sequences from the front- and back-vector in combination into the predetermined locus of a mammalian TI host cell's genome. RMCE can be implemented such that prokaryotic vector sequences are not introduced into the mammalian TI host cell's genome, thus, reducing and/or preventing unwanted triggering of host immune or defense mechanisms. The RMCE procedure can be repeated with multiple DNA sequences.

In certain embodiments of all aspects and embodiments, targeted integration is achieved by two RMCEs, wherein two different DNA sequences, each comprising at least one expression cassette encoding a part of a heteromultimeric polypeptide and/or at least one selection marker or part thereof flanked by two heterospecific RRSs, are both integrated into a predetermined site of the genome of a RRSs matching mammalian TI host cell. In certain embodiments, targeted integration is achieved by multiple RMCEs, wherein DNA sequences from multiple vectors, each comprising at least one expression cassette encoding a part of a heteromultimeric polypeptide and/or at least one selection marker or part thereof flanked by two heterospecific RRSs, are all integrated into a predetermined site of the genome of a mammalian TI host cell. In certain embodiments, the selection marker can be partially encoded on the first vector and partially encoded on the second vector such that only the correct integration of both by double RMCE allows for the expression of the selection marker.

In certain embodiments of all aspects and embodiments, targeted integration via recombinase-mediated recombination leads to the integration of the selection marker and/or the different expression cassettes for the nucleic acid according to the invention as well as the multimeric polypeptide into one or more predetermined integration sites of a host cell genome free of sequences from a prokaryotic vector.

In certain embodiments of all aspects and embodiments, a mammalian TI host cell comprises an integrated landing site, wherein the landing site comprises two or more recombination recognition sequences (RRS). The RRS can be recognized by a recombinase, for example, a Cre recombinase, an FLP recombinase, a Bxb1 integrase, or a φC31 integrase. The RRS can be selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. If multiple RRSs have to be present, the selection of each of the sequences is dependent on the other insofar as non-identical RRSs are chosen.

Generally, a mammalian TI host cell is a mammalian cell comprising a landing site integrated at a single site within a locus of the genome of the mammalian cell, wherein the landing site comprises a first and a second recombination recognition sequence flanking at least one first selection marker, and a third recombination recognition sequence located between the first and the second recombination recognition sequence, and all the recombination recognition sequences are different.

The selection marker(s) can be selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. The selection marker(s) can also be a fluorescent protein selected from the group consisting of green fluorescent protein (GFP), enhanced GFP (eGFP), a synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald6, CyPet, mCFPm, Cerulean, and T-Sapphire.

An exogenous nucleotide sequence is a nucleotide sequence that does not originate from a specific cell but can be introduced into said cell by DNA delivery methods, such as, e.g., by transfection, electroporation, or transformation methods. In certain embodiments, a mammalian TI host cell comprises at least one landing site integrated at one or more integration sites in the mammalian cell's genome. In certain embodiments, the landing site is integrated at one or more integration sites within a specific locus of the genome of the mammalian cell.

In certain embodiments of all aspects and embodiments, the integrated landing site comprises at least one selection marker. In certain embodiments, the integrated landing site comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments of all aspects and embodiments, a selection marker is located between a first and a second RRS and the two flanking RRSs are different. In certain preferred embodiments, the first flanking RRS is a LoxP L3 sequence (SEQ ID NO: 96) and the second flanking RRS is a LoxP 2L sequence (SEQ ID NO: 97). In certain embodiments, a LoxP L3 sequence is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientations.

In certain embodiments of all aspects and embodiments, the integrated landing site comprises a first and a second selection marker, which are flanked by two RRSs, wherein the first selection marker is different from the second selection marker. In certain embodiments, the two selection markers are both independently of each other selected from the group consisting of a glutamine synthetase selection marker, a thymidine kinase selection marker, a HYG selection marker, and a puromycin resistance selection marker. In certain embodiments, the integrated landing site comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, a mStrawberry, a J-red, a DsRed-monomer, an mOrange, an mKO, an mCitrine, a Venus, a YPet, an Emerald, a CyPet, an mCFPm, a Cerulean, and a T-Sapphire fluorescent protein. In certain embodiments, the first selection marker is a glutamine synthetase selection marker and the second selection marker is a GFP fluorescent protein. In certain embodiments, the two RRSs flanking both selection markers are different.

In certain embodiments of all aspects and embodiments, the selection marker is operably linked to a promoter sequence. In certain embodiments, the selection marker is operably linked to an SV40 promoter. In certain embodiments, the selection marker is operably linked to a human Cytomegalovirus (CMV) promoter.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located at a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′ end-independent manner.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

### Proteases

As used herein, the term “protease” and grammatical equivalents thereof denote enzymes that catalyze the hydrolysis of covalent peptide (amide) bonds. Proteases can be subdivided into different classes, such as, for example, serine proteases and matrix metalloproteinases.

Matrix metalloproteinases (MMP) are a family of metal-dependent, for example, Zn²⁺-dependent, endopeptidases. They preferably cleave components of the extracellular matrix. MMPs include collagenases, stromelysin, membrane-type metalloproteinases and gelatinases. In vivo MMPs occur as inactive precursors (zymogens), which need to be cleaved to obtain the catalytically active form. MMPs are specifically regulated by tissue inhibitors of matrix metalloproteinases.

Serine proteases are widely occurring in prokaryotes and eukaryotes. They are characterized by the presence of a catalytically active serine residue in the active center of the enzyme. Peptide bond cleavage by serine proteases encompasses the nucleophilic attack of the targeted peptide bond by the serine residue of the active center of the enzyme. Besides the serine residue further histidine and aspartate residues, respectively their side chains, can be involved. Together they form a so-called catalytic triad common to most serine proteases.

Serine proteases include, amongst others, chymotrypsin, trypsin, elastase, NS3, factor Xa, granzyme B, thrombin, plasmin, urokinase, tissue plasminogen activator, prostate-specific antigen; matrix metalloproteinases include, e.g., gelatinase B and gelatinase A.

Chymotrypsin acts on peptide bonds flanked with bulky hydrophobic amino acid residue, particularly phenylalanine, tryptophan and tyrosine.

Trypsin cleaves peptide bonds flanked with positively charged amino acid residues.

Elastase hydrolyzes peptide bonds flanked with small neutral/aliphatic amino acid residues, particularly alanine, methionine, glycine and valine.

### Self-Cleaving Peptides

The introduction of so-called self-cleaving peptide sequences between two coding sequences allows for the production of the two polypeptides flanking the self-cleaving peptide sequence in separated form from a single nucleic acid.

The incorporation of self-cleaving peptide sequences within a nucleic acid, and thereby in the corresponding mRNA, results in the skipping of or prevents the synthesis of a peptide bond at the C-terminus by the ribosome during protein synthesis (prevents the formation of peptide- or phosphodiester-bonds between amino acid residues or functions as pseudo stop-codon sequence that induces the translation complex to move from one codon to the next without forming a peptide bond). Thus, a single mRNA molecule encodes multiple different/separate proteins, which are generated by ribosome skipping during translation.

By the inclusion of a self-cleaving peptide two separate polypeptides are obtained, one corresponding to the sequence upstream of the self-cleaving peptide sequence and one corresponding to the sequence downstream of the self-cleaving peptide sequence. Although it is termed self-cleaving peptide sequence, actually none of the resulting products comprises the entire sequence but only the N-terminal or C-terminal part thereof, respectively. Use of the term “self-cleaving” is not intended to imply proteolytic activity.

Accordingly, the nucleic acids encoding the self-cleaving peptide sequence are arranged in such a way that they are between and in frame with the two coding regions preceding and succeeding it.

One type of self-cleaving peptide sequences are the viral 2-A self-cleaving peptide sequence. A detailed methodology for design and use of 2-A self-cleaving peptide sequences can be found in Szymczak-Workman et al. (Design and Construction of 2A Peptide-Linked Multi cistronic Vectors. Cold Spring Harb. Protoc. 2012 Feb. 1; 2012(2): 199-204), which is expressly incorporated by reference herein.

As with other self-cleaving peptide sequences, the incorporation of viral 2-A self-cleaving peptide sequence results in the ribosome skipping the synthesis of the peptide bond at the C-terminus of the 2-A self-cleaving peptide sequence. In more detail, the peptide bond linking the glycine amino acid residue and the proline amino acid residue at the C-terminus of the 2-A self-cleaving peptide sequence is not formed. Thereby two polypeptides are obtained, whereof one comprises a part of the 2-A self-cleaving peptide sequence at its C-terminus and the other at its N-terminus (see, e.g., Matsuzaki, J., et al., Sci. Rep. 5 (2015) 14896; Banu, N., et al., Sci. Rep. 4 (2014) 4166; Kim, et al., PLoS One 6 (2011) e18556; Donnelly, M. L., et al., J. Gen. Virol, 82 (2001) 1027-1101; Ryan, M. D., et al., J. Gen. Virol., 72 (2001) 2727-2732).

Exemplary viral 2-A self-cleaving peptides sequences are
the 2-A self-cleaving peptide sequence of Thosea asigna virus—termed T2A,
the 2-A self-cleaving peptide sequence of equine rhinitis A virus—termed E2A,
the 2-A self-cleaving peptide sequence of porcine teschovirus-1—termed P2A,
the 2-A self-cleaving peptide sequence of foot and mouth disease virus—termed F2A,
the 2-A self-cleaving peptide sequence of acute bee paralysis virus—A2A,
the 2-A self-cleaving peptide sequence of drosophila C virus—D2A,
the 2-A self-cleaving peptide sequence of infectious myonecrosis virus—I2A.

The following Table shows the sequences of members of the viral 2-A self-cleaving peptide sequence family. It is known from the art that by adding the peptidic linker sequence GSG (Gly-Ser-Gly) at the N-terminus of a 2-A self-cleaving peptide sequence (N-terminal to the 2-A self-cleaving peptide sequence) the cleavage efficiency can be increased. N-terminal to the 2-A self-cleaving peptide means that the sequence encoding GSG is upstream to the sequence encoding the 2-A self-cleaving peptide. Generally, the GSG will be immediately N-terminal to the 2-A self-cleaving peptide sequence. In certain embodiments, 1 to 10 other amino acid residues are inserted between the GSG and the 2-A self-cleaving peptide sequence. In certain embodiments, the polynucleotide sequence encoding GSG is GGC AGT GGA. As with any peptide-encoding polynucleotide, the nucleotide sequence may be altered without changing the encoded peptide sequence due to the degeneracy of the genetic code, as known to the person skilled in the art.

| self-cleaving | sequence (aa: amino acid |
|---|---|
| peptide | sequence; nt: exemplary coding nucleic acid sequence) |
| T2A | aa: EGRGSLLTCGDVEENPGP (SEQ ID NO: 14) |
| | nt: |
| | GAGGGCAGAGGAAGTCTGCTAACATGCGGTGACGTCG |
| | AGGAGAATCCTGGCCCA (SEQ ID NO: 15) |
| | aa: RAEGRGSLLTCGDVEENPGP (SEQ ID NO: 16) |
| | nt: |
| | AGGGCCGAGGGCAGAGGAAGTCTGCTAACATGCGGTG |
| | ACGTCGAGGAGAATCCTGGCCCA (SEQ ID NO: 17) |
| | aa: SGEGRGSLLTCGDVEENPGP (SEQ ID NO: 18) |
| | nt: |
| | TCCGGCGAGGGCAGAGGAAGTCTGCTAACATGCGGTGA |
| | CGTCGAGGAGAATCCTGGCCCA (SEQ ID NO: 19) |
| | aa: LEGGGEGRGSLLTCGDVEENPGPR (SEQ ID NO: 20) |
| | nt: |
| | CTGGAGGGCGGCGGCGAGGGCAGAGGAAGTCTGCTAA |
| | CATGCGGTGACGTCGAGGAGAATCCTGGCCCAAGG |
| | (SEQ ID NO: 21) |
| P2A | aa: LLKQAGDVEENPGP (SEQ ID NO: 22) |
| | nt: |
| | CTGCTGAAGCAGGCCGGCGACGTGGAGGAGAACCCCG |
| | GCCCC (SEQ ID NO: 23) |
| | aa: ATNFSLLKQAGDVEENPGP (SEQ ID NO: 24) |
| | nt: |
| | GCCACCAACTTCTCCCTGCTGAAGCAGGCCGGCGACGT |
| | GGAGGAGAACCCCGGCCCC (SEQ ID NO: 25) |
| | aa: SGATNFSLLKQAGDVEENPGP (SEQ ID NO: 26) |
| | nt: |
| | TCCGGCGCCACCAACTTCTCCCTGCTGAAGCAGGCCGG |
| | CGACGTGGAGGAGAACCCCGGCCCC (SEQ ID NO: 27) |
| E2A | aa: LLKLAGDVESNPGP (SEQ ID NO: 28) |
| | nt: |
| | CTGCTGAAGCTGGCCGGCGACGTGGAGTCCAACCCCGG |
| | CCCC (SEQ ID NO: 29) |
| | aa: QCTNYALLKLAGDVESNPGP (SEQ ID NO: 30) |
| | nt: |
| | CAGTGCACCAACTACGCCCTGCTGAAGCTGGCCGGCGA |
| | CGTGGAGTCCAACCCCGGCCCC (SEQ ID NO: 31) |
| | aa: SGQCTNYALLKLAGDVESNPGP (SEQ ID NO: 32) |
| | nt: |
| | TCCGGCCAGTGCACCAACTACGCCCTGCTGAAGCTGGC |
| | CGGCGACGTGGAGTCCAACCCCGGCCCC (SEQ ID NO: 33) |
| F2A | aa: NFDLLKLAGDVESNPGP (SEQ ID NO: 34) |
| | nt: |
| | AACTTCGACCTGCTGAAGCTGGCCGGCGACGTGGAGTC |
| | CAACCCCGGCCCC (SEQ ID NO: 35) |
| | aa: LNFDLLKLAGDVESNPGP (SEQ ID NO: 36) |
| | nt: |
| | CTGAACTTCGACCTGCTGAAGCTGGCCGGCGACGTGGA |
| | GTCCAACCCCGGCCCC (SEQ ID NO: 37) |
| | aa: LLNFDLLKLAGDVESNPGP (SEQ ID NO: 38) |
| | nt: |
| | CTGCTGAACTTCGACCTGCTGAAGCTGGCCGGCGACGT |
| | GGAGTCCAACCCCGGCCCC (SEQ ID NO: 39) |
| | aa: TLNFDLLKLAGDVESNPGP (SEQ ID NO: 40) |
| | nt: |
| | ACCCTGAACTTCGACCTGCTGAAGCTGGCCGGCGACGT |
| | GGAGTCCAACCCCGGCCCC (SEQ ID NO: 41) |
| | aa: QLLNFDLLKLAGDVESNPGP (SEQ ID NO: 42) |
| | nt: |
| | CAGCTGCTGAACTTCGACCTGCTGAAGCTGGCCGGCGA |
| | CGTGGAGTCCAACCCCGGCCCC (SEQ ID NO: 43) |
| | aa: VKQTLNFDLLKLAGDVESNPGP (SEQ ID NO: 44) |
| | nt: |
| | GTGAAACAGACTTTGAATTTTGACCTTCTCAAGTTGGCG |
| | GGAGACGTGGAGTCCAACCCAGGGCCG (SEQ ID NO: 45) |
| | aa: APVKQTLNFDLLKLAGDVESNPGP (SEQ ID NO: 46) |
| | nt: |
| | GCCCCCGTGAAGCAGACCCTGAACTTCGACCTGCTGAA |
| | GCTGGCCGGCGACGTGGAGTCCAACCCCGGCCCC |
| | (SEQ ID NO: 47) |
| | aa: SGVKQTLNFDLLKLAGDVESNPGP (SEQ ID NO: 48) |
| | nt: |
| | TCCGGCGTGAAGCAGACCCTGAACTTCGACCTGCTGAA |
| | GCTGGCCGGCGACGTGGAGTCCAACCCCGGCCCC |
| | (SEQ ID NO: 49) |
| | aa: EARHKQKIVAPVKQTLNFDLLKLAGDVESNPGP |
| | (SEQ ID NO: 50) |
| | nt: |
| | GAGGCCAGGCACAAGCAGAAGATCGTGGCCCCCGTGA |
| | AGCAGACCCTGAACTTCGACCTGCTGAAGCTGGCCGGC |
| | GACGTGGAGTCCAACCCCGGCCCC (SEQ ID NO: 51) |
| | aa: |
| | LLAIHPTEARHKQKIVAPVKQTLNFDLLKLAGDVESNPGP |
| | (SEQ ID NO: 52) |
| | nt: |
| | CTGCTGGCCATCCACCCCACCGAGGCCAGGCACAAGCA |
| | GAAGATCGTGGCCCCCGTGAAGCAGACCCTGAACTTCG |
| | ACCTGCTGAAGCTGGCCGGCGACGTGGAGTCCAACCCC |
| | GGCCCC (SEQ ID NO: 53) |
| A2A | aa: GSWTDILLLLSGDVETNPGP (SEQ ID NO: 54) |
| | nt: |
| | GGCTCCTGGACCGACATCCTGCTGCTGCTGTCCGGCGA |
| | CGTGGAGACCAACCCCGGCCCC (SEQ ID NO: 55) |
| | aa: TGFLNKLYHCGSWTDILLLLSGDVETNPGP (SEQ ID NO: 56) |
| | nt: |
| | ACCGGCTTCCTGAACAAGCTGTACCACTGCGGCTCCTG |
| | GACCGACATCCTGCTGCTGCTGTCCGGCGACGTGGAGA |
| | CCAACCCCGGCCCC (SEQ ID NO: 57) |
| D2A | aa: AARQMLLLLSGDVETNPGP (SEQ ID NO: 58) |
| | nt: |
| | GCCGCCAGGCAGATGCTGCTGCTGCTGTCCGGCGACGT |
| | GGAGACCAACCCCGGCCCC (SEQ ID NO: 59) |
| I2A | aa: WDPTYIEISDCMLPPPDLTSCGDVESNPGP (SEQ ID NO: 60) |
| | nt: |
| | TGGGACCCCACCTACATCGAGATCTCCGACTGCATGCT |
| | GCCCCCCCCCGACCTGACCTCCTGCGGCGACGTGGAGT |
| | CCAACCCCGGCCCC (SEQ ID NO: 61) |
| | aa: RDVRYIEKPFDKEEHTDILLSGDVEENPGP (SEQ ID NO: 62) |
| | nt: |
| | AGGGACGTGAGGTACATCGAGAAGCCCTTCGACAAGG |
| | AGGAGCACACCGACATCCTGCTGTCCGGCGACGTGGAG |
| | GAGAACCCCGGCCCC (SEQ ID NO: 63) |
| artificial | aa: SSIIRTKMLVSGDVEENPGP (SEQ ID NO: 64) |
| | nt: |
| | TCCTCCATCATCAGGACCAAGATGCTGGTGTCCGGCGA |
| | CGTGGAGGAGAACCCCGGCCCC (SEQ ID NO: 65) |
| | aa: AKFQIDKILISGDVELNPGP (SEQ ID NO: 66) |
| | nt: |
| | GCCAAGTTCCAGATCGACAAGATCCTGATCTCCGGCGA |
| | CGTGGAGCTGAACCCCGGCCCC (SEQ ID NO: 67) |
| | aa: GATNFSLLKLAGDVELNPGP (SEQ ID NO: 68) |
| | nt: |
| | GGCGCCACCAACTTCTCCCTGCTGAAGCTGGCCGGCGA |
| | CGTGGAGCTGAACCCCGGCCCC (SEQ ID NO: 69) |
| | aa: SKFQIDKILISGDIELNPGP (SEQ ID NO: 70) |
| | nt: |
| | TCCAAGTTCCAGATCGACAAGATCCTGATCTCCGGCGA |
| | CATCGAGCTGAACCCCGGCCCC (SEQ ID NO: 71) |
| | aa: KAVRGYHADYYKQRLIHDVEMNPGP (SEQ ID NO: 72) |
| | nt: |
| | AAGGCCGTGAGGGGCTACCACGCCGACTACTACAAGCA |
| | GAGGCTGATCCACGACGTGGAGATGAACCCCGGCCCC |
| | (SEQ ID NO: 73) |
| | aa: CDAQRQKLLLSGDIEQNPGP (SEQ ID NO: 74) |
| | nt: |
| | TGCGACGCCCAGAGGCAGAAGCTGCTGCTGTCCGGCGA |
| | CATCGAGCAGAACCCCGGCCCC (SEQ ID NO: 75) |
| | aa: FLRKRTQLLMSGDVESNPGP (SEQ ID NO: 76) |
| | nt: |
| | TTCCTGAGGAAGAGGACCCAGCTGCTGATGTCCGGCGA |
| | CGTGGAGTCCAACCCCGGCCCC (SEQ ID NO: 77) |
| | aa: TRAEIEDELIRAGIESNPGP (SEQ ID NO: 78) |
| | nt: |
| | ACCAGGGCCGAGATCGAGGACGAGCTGATCAGGGCCG |
| | GCATCGAGTCCAACCCCGGCCCC (SEQ ID NO: 79) |
| | aa: HYAGYFADLLIHDIETNPGP (SEQ ID NO: 80) |
| | nt: |
| | CACTACGCCGGCTACTTCGCCGACCTGCTGATCCACGA |
| | CATCGAGACCAACCCCGGCCCC (SEQ ID NO: 81) |
| | aa: GIFNAHYAGYFADLLIHDIETNPGP (SEQ ID NO: 82) |
| | nt: |
| | GGCATCTTCAACGCCCACTACGCCGGCTACTTCGCCGA |
| | CCTGCTGATCCACGACATCGAGACCAACCCCGGCCCC |
| | (SEQ ID NO: 83) |
| | aa: |
| | VTELLYRMKRAETYCPRPLLAIHPTEARHKQKIVAPVKQT |
| | (SEQ ID NO: 84) |
| | nt: |
| | GTGACCGAGCTGCTGTACAGGATGAAGAGGGCCGAGA |
| | CCTACTGCCCCAGGCCCCTGCTGGCCATCCACCCCACC |
| | GAGGCCAGGCACAAGCAGAAGATCGTGGCCCCCGTGA |
| | AGCAGACC (SEQ ID NO: 85) |

Substitution of amino acid residues is within the skill of those in the art. Thus, the term “2-A self-cleaving peptide sequence” encompasses variants of the foregoing peptides that retain the desired skipping/self-cleavage activity but, optionally, have 1, 2, 3, 4, 5, 6, 7, 8, 9, 10 or more substitutions relative to the wild-type 2-A self-cleaving peptide sequence (see, e.g., Liu et al., Sci. Rep. 7 (2017) 2193).

In certain embodiments of all aspects and embodiments, the 2-A self-cleaving peptide sequence is a variant of a wild-type viral 2-A self-cleaving peptide sequence, i.e. of SEQ ID NO: 14-63. Such a variant has at least 80%, at least 85%, at least 90%, at least 95%, at least 96%, at least 97%, at least 98%, at least 99%, or more, sequence identity to a wild-type viral 2-A self-cleaving peptide sequence and retains the ribosome skipping function. In certain embodiments, at least one N-terminal amino acid or the respective codon of any one of SEQ ID NO: 14-85 is deleted, for example 1, 2, 3, 4 or 5 amino acids or the respective number of codons, including ranges between any two of the listed values, are deleted. In certain embodiments, at least one C-terminal amino acid or the respective codon of any one of SEQ ID NO: 14-85 is deleted, for example, 1, 2, 3, 4 or 5 amino acids, or the respective number of codons, including ranges between any two of the listed values, are deleted. In certain embodiments, at least 1, 2, 3, 4 or 5 amino acids or the respective number of codons, including ranges between any two of the listed values, of any one of SEQ ID NO: 14-85 are substituted, for example as conservative amino acid substitutions.

In certain embodiments of all aspects and embodiments, the T2A self-cleaving peptide sequence comprises an amino acid sequence comprising EGRGSLLTCGDVEENPGP (SEQ ID NO: 14) or a sequence having at least 70%, 80%, 90%, 95%, or 99% sequence identity to the amino acid sequence comprising EGRGSLLTCGDVEENPGP (SEQ ID NO: 14) and having ribosome skipping function.

In certain embodiments of all aspects and embodiments, the GSG-T2A self-cleaving peptide sequence comprises an amino acid sequence comprising GSGEGRGSLLTCGDVEENPGP (SEQ ID NO: 108) or a sequence having at least 70%, 80%, 90%, 95%, or 99% sequence identity to the amino acid sequence comprising GSGEGRGSLLTCGDVEENPGP (SEQ ID NO: 108) and having ribosome skipping function. In certain embodiments, the GSG-T2A self-cleaving peptide sequence is encoded by a nucleic acid sequence comprising

(SEQ ID NO: 109)
GGCAGTGGAGAGGGCAGAGGAAGTCTGCTAACATGCGGTGACGTCGAGG
AGAATCCTGGCCCA.

In certain embodiments of all aspects and embodiments, the E2A self-cleaving peptide sequence comprises an amino acid sequence comprising QCTNYALLKLAGDVESNPGP (SEQ ID NO: 30) or a sequence having at least 70%, 80%, 90%, 95%, or 99% identity to the amino acid sequence comprising QCTNYALLKLAGDVESNPGP (SEQ ID NO: 30) and ribosome skipping function. In certain embodiments, the GSG-E2A self-cleaving peptide sequence comprises an amino acid sequence comprising GSGQCTNYALLKLAGDVESNPGP (SEQ ID NO: 110) or a sequence having at least 70%, 80%, 90%, 95%, or 99% identity to the amino acid sequence comprising GSGQCTNYALLKLAGDVESNPGP (SEQ ID NO: 110) and having ribosome skipping function.

In certain embodiments of all aspects and embodiments, the F2A self-cleaving peptide comprises an amino acid sequence comprising VKQTLNFDLLKLAGDVESNPGP (SEQ ID NO: 44) or a sequence having at least 70%, 80%, 90%, 95%, or 99% identity to the amino acid sequence comprising VKQTLNFDLLKLAGDVESNPGP (SEQ ID NO: 44) and having ribosome skipping function. In certain embodiments, the GSG-F2A self-cleaving peptide comprises an amino acid sequence comprising GSGVKQTLNFDLLKLAGDVESNPGP (SEQ ID NO: 112) or a sequence having at least 70%, 80%, 90%, 95%, or 99% identity to the amino acid sequence comprising GSGVKQTLNFDLLKLAGDVESNPGP (SEQ ID NO: 112) and having ribosome skipping function.

In certain embodiments of all aspects and embodiments, the P2A self-cleaving peptide comprises an amino acid sequence comprising ATNFSLLKQAGDVEENPGP (SEQ ID NO: 24) or a sequence having at least 70%, 80%, 90%, 95%, or 99% identity to the amino acid sequence comprising ATNFSLLKQAGDVEENPGP (SEQ ID NO: 24) and having ribosome skipping function. In certain embodiments, the GSG-P2A self-cleaving peptide comprises an amino acid sequence comprising GSGATNFSLLKQAGDVEENPGP (SEQ ID NO: 114) or a sequence having at least 70%, 80%, 90%, 95%, or 99% identity to the amino acid sequence comprising GSGATNFSLLKQAGDVEENPGP (SEQ ID NO: 114) and having ribosome skipping function.

In certain embodiments of all aspects and embodiments, between 1 and 5, or more than 5, Gly or Ser residues are added/inserted N- and/or C-terminally to the 2-A self-cleaving peptide sequence. In certain embodiments, the amino acid residues GSG are added N- and/or C-terminally to the 2-A self-cleaving peptide sequence

In certain embodiments of all aspects and embodiments, the 2-A self-cleaving peptide sequence is combined at its N- or C-terminus with a protease cleavage site, for example a furin cleavage site (RKRR (SEQ ID NO: 116); cgcaaacggaga SEQ ID NO: 117).

### Peptidic Linker

In certain embodiments of all aspects and embodiments, the recombinant polypeptide comprises a protease-cleavable site. In certain embodiments the protease cleavable site is comprised in a peptidic linker (used interchangeably with cleavable peptidic linker). In certain embodiments, a peptidic linker that is a cleavable linker or that is protease-cleavable is a peptidic linker that comprises at least one peptide bond which, in one preferred embodiment, is within a recognition (amino acid) sequence (recognition site) of a protease. In certain embodiments, a cleavable peptidic linker is a target substrate for a protease, such that it is preferentially or specifically cleaved by a protease compared to a peptidic linker that does not contain a recognition sequence for the same protease.

In certain embodiments of all aspects and embodiments, the cleavable peptidic linker comprises a recognition sequence or cleavage site for a particular protease. A recognition (amino acid) sequence is the sequence recognized by the active site of the protease and wherein a peptide bond is cleaved by the protease.

For example, for a serine protease, a recognition sequence is made up of the amino acid residues N-terminal and C-terminal to the peptide bond that is cleaved. These residues are denoted as P4-P1 (N-terminal) and P1′-P4′ (C-terminal) amino acid residues. The cleavage occurs after the P1 position, i.e. the peptide bond between amino acid residue P1 and P1′ is cleaved. Generally, the recognition sequence for a serine protease is six to eight amino acid residues in length, but can be longer or even shorter depending upon the specific protease. Typically, the cleavable peptidic linker includes a P1-P1′ cleavable bond within a recognition sequence that is recognized by a protease.

In certain embodiments of all aspects and embodiments, the cleavable peptidic linker is one whose cleavage by a protease is substantially higher than cleavage of a non-target substrate of the same protease. Typically, a protease exhibits specificity (preference) for cleavage of a particular polypeptide comprising the respective recognition sequence compared to another polypeptide not comprising the recognition sequence. Such specificity can be determined based on the rate constant of cleavage of a sequence, e.g. peptidic linker sequence. Said rate constant is a value reflecting the specificity of the protease for its substrate as well as its efficiency. Any method to determine the rate constant for cleavage can be used. For example, a substrate comprising the respective recognition sequence is conjugated to a fluorogenic moiety, which is released upon cleavage by the protease. By determining the rate of cleavage at different protease concentrations the rate constant for cleavage (kcat/Km) can be determined with respect to the combination of a particular protease and a particular substrate. In certain embodiments, a cleavable peptidic linker is a peptidic linker that is cleaved by a protease at a rate of more than 1×10⁷ M⁻¹S, or more than 10⁸ M⁻¹S, more than 1×10⁹ M⁻¹S, or more than 1×10¹⁰ M⁻¹S.

In certain embodiments of all aspects and embodiments, at least one polypeptide of the multispecific antibody produced with a recombinant cell according to the invention comprises a recognition sequence for a protease that include, for example, matrix metalloproteases (MMP), cysteine proteases, serine proteases and plasmin activators. In certain embodiments, the polypeptide comprises a recognition sequence for a protease that is a protease that is produced by a tumor, an activated immune effector cell (e.g. a T-cell or a NK cell), or a cell in a tumor microenvironment.

In certain embodiments of all aspects and embodiments, the recombinant antibody comprises at least one polypeptide comprising a recognition sequence that is specifically recognized by one or more of the following enzymes or proteases: ADAMS; ADAMTS; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKs, e.g., KLK4, KLK5, KLK6, KLK7, KLK8, KLK10, KLK11, KLK13, or KLK14; Metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27; serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4.

In certain embodiments of all aspects and embodiments, the recombinant antibody comprises at least one polypeptide comprising a cleavable peptidic linker that comprises an amino acid sequence that is recognized and cleaved by Granzyme B. In certain embodiments, the recombinant antibody comprises at least one polypeptide comprising a cleavable peptidic linker that comprises an amino acid sequence having the general formula P4 P3 P2 P1JP1′, wherein P4 is amino acid I, L, Y, M, F, V, or A; P3 is amino acid A, G, S, V, E, D, Q, N, or Y; P2 is amino acid H, P, A, V, G, S, or T; P1 is amino acid D or E; and P1′ is amino acid I, L, Y, M, F, V, T, S, G or A. In certain embodiments, the recombinant antibody comprises at least one polypeptide comprising a cleavable peptidic linker that comprises an amino acid sequence having the general formula P4 P3 P2 P1JP1′, wherein P4 is amino acid I or L; P3 is amino acid E; P2 is amino acid P or A; P1 is amino acid D; and P1′ is amino acid I, V, T, S, or G.

In certain embodiments of all aspects and embodiments, the recombinant antibody comprises at least one polypeptide comprising a cleavable peptidic linker that comprises the amino acid sequence LEAD (SEQ ID NO: 119), LEPD (SEQ ID NO: 120), or LEAE (SEQ ID NO: 121). In certain embodiments, the recombinant antibody comprises at least one polypeptide comprising a cleavable peptidic linker that comprises the amino acid sequence IEPDI (SEQ ID NO: 122), LEADT (SEQ ID NO: 123), IEPDG (SEQ ID NO: 124), IEPDV (SEQ ID NO: 125), IEPDS (SEQ ID NO: 126), IEPDT (SEQ ID NO: 127), IEPDP (SEQ ID NO: 128), LEPDG (SEQ ID NO: 129) or LEADG (SEQ ID NO: 130).

In certain embodiments of all aspects and embodiments, the recombinant antibody comprises at least one polypeptide comprising a cleavable peptidic linker that comprises an amino acid that is a substrate for matriptase.

In certain embodiments, the recombinant antibody comprises at least one polypeptide comprising a cleavable peptidic linker that comprises the sequence P1QAR↓(A/V/K), wherein P1 is any amino acid.

In certain embodiments, the recombinant antibody comprises at least one polypeptide comprising a cleavable peptidic linker that comprises the sequence RQAR(A/V/K) (SEQ ID NO: 181 with P1=R). In certain embodiments, the recombinant antibody comprises at least one polypeptide comprising a cleavable peptidic linker that comprises the amino acid sequence RQAR (SEQ ID NO: 132). In certain embodiments, the recombinant antibody comprises at least one polypeptide comprising a cleavable peptidic linker that comprises the amino acid sequence RQARK (SEQ ID NO: 133).

In certain embodiments, the recombinant antibody comprises at least one polypeptide comprising a cleavable peptidic linker that comprises the sequence PQAR(A/V/K) (SEQ ID NO: 182 with P1=P). In certain embodiments, the recombinant antibody comprises at least one polypeptide comprising a cleavable peptidic linker that comprises the amino acid sequence PQAR (SEQ ID NO: 166). In one preferred embodiment, the recombinant antibody comprises at least one polypeptide comprising a cleavable peptidic linker that comprises the amino acid sequence PQARK (SEQ ID NO: 167).

In certain embodiments, the recombinant antibody comprises at least one polypeptide comprising a cleavable peptidic linker that comprises the sequence HQAR(A/V/K) (SEQ ID NO: 183 with P1=H). In certain embodiments, the recombinant antibody comprises at least one polypeptide comprising a cleavable peptidic linker that comprises the amino acid sequence HQAR (SEQ ID NO: 168). In one preferred embodiment, the recombinant antibody comprises at least one polypeptide comprising a cleavable peptidic linker that comprises the amino acid sequence HQARK (SEQ ID NO: 169).

In certain embodiments, the recombinant antibody comprises at least one polypeptide comprising a cleavable peptidic linker that comprises the sequence P1MAK↓(A/V/K), wherein P1 is any amino acid. In certain embodiments, the recombinant antibody comprises at least one polypeptide comprising a cleavable peptidic linker that comprises the sequence PMAK(A/V/K) (SEQ ID NO: 171 with P1=P). In certain embodiments, the recombinant antibody comprises at least one polypeptide comprising a cleavable peptidic linker that comprises the amino acid sequence PMAK (SEQ ID NO: 172). In one preferred embodiment, the recombinant antibody comprises at least one polypeptide comprising a cleavable peptidic linker that comprises the amino acid sequence PMAKK (SEQ ID NO: 173).

In certain embodiments of all aspects and embodiments, the recombinant antibody comprises at least one polypeptide comprising a cleavable peptidic linker that comprises an amino acid that is a substrate for one or more matrix metalloproteases (MMPs). In certain embodiments, the MMP is MMP-2. In certain embodiments, the recombinant antibody comprises at least one polypeptide comprising a cleavable peptidic linker that comprises a sequence having the general formula P3 P2 P1↓P1′, wherein P3 is P, V or A; P2 is Q or D; P1 is A or N; and P1′ is L, I or M. In certain embodiments, the recombinant antibody comprises at least one polypeptide comprising a cleavable peptidic linker that comprises the general formula P3 P2 P1↓P1′, wherein P3 is P; P2 is Q or D; P1 is A or N; and P1′ is L or I. In certain embodiments, the recombinant antibody comprises at least one polypeptide comprising a cleavable peptidic linker that comprises the amino acid sequence PAGL (SEQ ID NO: 135).

In certain embodiments of all aspects and embodiments, the recombinant antibody comprises at least one polypeptide comprising a cleavable peptidic linker that comprises the amino acid sequence TGLEADGSPAGLGRQARVG (SEQ ID NO: 136); TGLEADGSRQARVGPAGLG (SEQ ID NO: 137); TGSPAGLEADGSRQARVGS (SEQ ID NO: 138); TGPAGLGLEADGSRQARVG (SEQ ID NO: 139); TGRQARVGLEADGSPAGLG (SEQ ID NO: 140); TGSRQARVGPAGLEADGS (SEQ ID NO: 141); and TGPAGLGSRQARVGLEADGS (SEQ ID NO: 142); GPAGLGLEPDGSRQARVG (SEQ ID NO: 143); GGSGGGGIEPDIGGSGGS (SEQ ID NO: 144); GGSGGGGLEADTGGSGGS (SEQ ID NO: 145); GSIEPDIGS (SEQ ID NO: 146); GSLEADTGS (SEQ ID NO: 147); GGSGGGGIEPDGGGSGGS (SEQ ID NO: 148); GGSGGGGIEPDVGGSGGS (SEQ ID NO: 149); GGSGGGGIEPDSGGSGGS (SEQ ID NO: 150); GGSGGGGIEPDTGGSGGS (SEQ ID NO: 151); GGGSLEPDGSGS (SEQ ID NO: 152); GPAGLGLEADGSRQARVG (SEQ ID NO: 153), GGEGGGGSGGSGGGS (SEQ ID NO: 154); GSSAGSEAGGSGQAGVGS (SEQ ID NO: 155); GGSGGGGLEAEGSGGGGS (SEQ ID NO: 156); GGSGGGGIEPDPGGSGGS(SEQ ID NO: 157); TGGSGGGGIEPDIGGSGGS (SEQ ID NO: 158).

### Protease Inhibitors

In certain embodiments of all aspects and embodiments, the protease inhibitor is selected from C1-protease inhibitors, trypsin inhibitors, thrombin inhibitors, antithrombin-III (AT-III), heparin-cofactor-II, BPTI, aprotinin, pepstatin, leupeptin and epsilon-aminocaproic acid. In one preferred embodiment, the protease inhibitor is BPTI.

In certain embodiments of all aspects and embodiments, the protease inhibitor inhibits elastase and comprises the sequence Ala-Ala-Pro-Val (SEQ ID NO: 159); inhibits elastase, and comprises the general structure AA1-AA2-AA3-AA4, in which AA1 is -Arg-, -Phe- and -Ile- or is a bond; AA2 is -Ala-, -Phe-, -Cit- and -Nle-; AA3 is -Trp-, -Val- and -Tyr-; and AA4: -Phe- and -Gly-; inhibits elastase, and comprises a constrained or β-hairpin peptide as shown in U.S. Pat. No. 8,658,604; or inhibits elastase, and comprises Pep4 (KRCCPDTCGIKCL; SEQ ID NO: 161) or Pep4M (KRMMPDTMGIKML; SEQ ID NO: 162).

In certain embodiments of all aspects and embodiments, the protease inhibitor inhibits matrix metalloprotease and comprises the sequence of an inhibitory peptide reported in Ndinguri et al., Molecules 17 (2012) 14230-14248.

In certain embodiments of all aspects and embodiments, the protease inhibitor inhibits cathepsin and comprises the structure Z-Phe-Gly-NHO-Bz (Z=carboxybenzyl, Bz=benzyl); or inhibits cathepsin and comprises the structure Z-Phe-Phe-DK (SEQ ID NO: 163) or Z-Phe-Phe-CHN₂.

In certain embodiments of all aspects and embodiments, the protease inhibitor inhibits chymase and comprises the structure Z-Arg-Glu-Thr-Phep(OPh)₂.

In certain embodiments of all aspects and embodiments, the protease inhibitor inhibits thrombin and/or coagulation factors IX and X and is selected from hirudin (MTYTDCTESGQNLCLCEGSNVCGQGNKCILGSDGEKNQCVTGEGTPKPQSHNDGDF EEIPEEYLQ; SEQ ID NO: 165) or a derivative thereof, such as lepirudin or desirudin.

In certain embodiments of all aspects and embodiments according to the invention, the protease inhibitor inhibits plasminogen activator and comprises bovine pancreatic trypsin inhibitor (BPTI) (SEQ ID NO: 86; AQRPDFCLEPPYTGPCKARMIRYFYNAKAGLCQPFVYGGCRAKRNNFKSSEDCMRT CGGA) or the plasminogen activator inhibitor type 1 (PAI-1)-derived peptide EEIIMD (SEQ ID NO: 88).

The term “proteinaceous protease inhibitor” as used herein denotes inhibitors of naturally occurring proteases that can be recombinantly produced. In certain embodiments of all aspects and embodiments according to the current invention, the proteinaceous protease inhibitor is a serine protease inhibitor. In certain embodiments of all aspects and embodiments according to the current invention, the proteinaceous protease inhibitor is a trypsin inhibitor. In certain preferred embodiments of all aspects and embodiments according to the current invention, the proteinaceous protease inhibitor is a pancreatic trypsin inhibitor. In one preferred embodiment of all aspects and embodiments according to the current invention, the proteinaceous protease inhibitor is bovine pancreatic trypsin inhibitor (BPTI). Said BPTI has a mature amino acid sequence of SEQ ID NO: 86, which is generated from the pro-form of SEQ ID NO: 177 by processing during expression and secretion.

The following examples, sequences and figures are provided to aid the understanding of the present invention, the true scope of which is set forth in the appended claims. It is understood that modifications can be made in the procedures set forth without departing from the spirit of the invention.

### DESCRIPTION OF THE EXAMPLES

### Example 1

### Generation of Expression Plasmids:

### a) Common-Light Chain Bispecific Antibody in TCB Format

As recombinant polypeptide/protein a bispecific antibody consisting of two different immunoglobulin heavy chains (denoted as “K” (knob chain) and “H” (hole chain), respectively) and a common light chain (denoted as “L”) was used. For the heterodimerization of the heavy chains the knob-into-hole technology was used. The fully assembled bispecific antibody comprises three copies of the light chain. Consequently the stoichiometry of the K:H:L chains of the fully assembled antibody is 1:1:3.

The expression cassette configuration (including the Cre-recombinase sites) after targeted stable integration into the host cell's genome was either L3-K-L-L-LoxFas-H-L-2L (used for generating the data presented in FIGS. 2 to 7) or L3-K-K-L-L-LoxFas-K-L-H-2L (used for generating the data presented in FIG. 8 and Table 2). L3 (SEQ ID NO: 96), LoxFas (SEQ ID NO: 98) and 2L (SEQ ID NO: 97) are the heterospecific loxP sites used for targeted integration.

### Expression Cassettes

For the expression of an immunoglobulin chain a transcription unit comprising the following functional elements was used:
the immediate early enhancer and promoter from the human cytomegalovirus including intron A (CMV promoter) (SEQ ID NO: 103),
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA) (SEQ ID NO: 100), and
the human gastrin terminator (hGT) (SEQ ID NO: 101).

Beside the expression cassette of the immunoglobulin subunit the shuttle plasmid contained
an origin of replication from the vector pUC18 which allows replication of this plasmid in E. coli, and
a beta-lactamase gene which confers ampicillin resistance in E. coli.

### Front Vector

DNA fragments coding for K and L were chemically synthesized and introduced into shuttle plasmids using suitable restriction sites, thereby yielding the complete expression cassettes for K and L. The expression cassettes for K and L were excised from their shuttle plasmids in a way that provided them with suitable sticky ends.

A first front vector was generated by combining one DNA fragment carrying the expression cassette for K and two fragments carrying the expression cassette for L with a fourth fragment carrying the front vector backbone elements in a four-way ligation reaction. The expression cassette configuration (including the Cre-recombinase sites) of this final front vector was L3-K-L-L-LoxFas.

Alternatively, a second front vector was generated by combining two DNA fragments carrying the expression cassette for K and two fragments carrying the expression cassette for L with a fifth fragment carrying the front vector backbone elements in a five-way ligation reaction. The expression cassette configuration (including the Cre-recombinase sites) of this final front vector was L3-K-K-L-L-LoxFas.

Beside the expression cassettes of K and L the front vector each contained
an SV40 enhancer and early promoter (SEQ ID NO: 102) with a start codon upstream of (or in other words, 5′ to) LoxFas to drive resistance marker expression after stable integration,
an origin of replication from the vector pUC18 which allows replication of this plasmid in E. coli, and
a beta-lactamase gene which confers ampicillin resistance in E. coli.

### Back Vector

A DNA fragment coding for H was chemically synthesized and introduced into a shuttle plasmid using suitable restriction sites, thereby yielding the complete expression cassette for H. The expression cassettes for H as well as K and L (see front vector section above) were excised from their shuttle plasmids in a way that provided them with suitable sticky ends.

A first back vector was generated by combining one DNA fragment carrying the expression cassette for H and one fragment carrying the expression cassette for L with a third fragment carrying the back plasmid backbone elements in a three-way ligation reaction. The expression cassette configuration (including the Cre-recombinase sites) of this final back vector was LoxFas-H-L-2L.

Alternatively, a second back vector was generated by combining one fragment carrying the expression cassette for K, one fragment carrying the expression cassette for H and one fragment carrying the expression cassette for L with a fourth fragment carrying the back vector backbone in a four-way ligation. In this case the expression cassette configuration (including the Cre-recombinase sites) of the final back vector was LoxFas-K-H-L-2L.

Beside the immunoglobulin expression cassettes the back vector each contained
a sequence encoding puromycin acetyltransferase (SEQ ID NO: 02) lacking the start codon directly downstream of (or in other words, 3′ to) the LoxFas site,
an origin of replication from the vector pUC18 which allows replication of this plasmid in E. coli, and
a beta-lactamase gene which confers ampicillin resistance in E. coli.

### b) HAI Expression Constructs

In order to evaluate the effect of the protease inhibitor HAI-1 on the expression of the bispecific antibody described under a), vectors for the additional expression of HAI-1 from a separate expression cassette were generated. The expression cassette configuration (including the Cre-recombinase sites) after targeted stable integration into the host genome was L3-K-L-L-LoxFas-H-L-HAI-1-2L.

### Expression Cassettes

The same immunoglobulin expression cassettes were used as described in Example 1 a).

For the expression of HAI-1 different expression cassettes were generated. These contained either
the immediate early enhancer and promoter from the human cytomegalovirus including intron A (CMV promoter) (SEQ ID NO: 103),
a human heavy chain immunoglobulin 5′-untranslated region (5′UTR),
a nucleic acid encoding soluble HAI-1 (SEQ ID NO: 180),
the bovine growth hormone polyadenylation sequence (BGH pA) (SEQ ID NO: 100), and
the human gastrin terminator (hGT) (SEQ ID NO: 101); or
the enhancer and early promoter of simian virus 40 (SV40) (SEQ ID NO: 102),
a nucleic acid encoding soluble HAI-1 (SEQ ID NO: 180),
the bovine growth hormone polyadenylation sequence (BGH pA) (SEQ ID NO: 100), and
the human gastrin terminator (hGT) (SEQ ID NO: 101).

Beside the HAI-1 expression cassette the vector each contained
an origin of replication from the vector pUC18 which allows replication of this plasmid in E. coli, and
a beta-lactamase gene which confers ampicillin resistance in E. coli.

### Front Vector

The front vector with the expression cassette configuration (including the Cre-recombinase sites) L3-K-L-L-LoxFas described in Example 1 a) was used.

### Back Vector

DNA fragments encoding HAI-1 were chemically synthesized and introduced into shuttle plasmids using suitable restriction sites, thereby yielding the complete expression cassettes for HAI-1. The expression cassettes for HAI-1 were excised from their shuttle plasmids in a way that provided them with suitable sticky ends.

The final back vector was generated by combining one DNA fragment carrying the expression cassette for H (see Example 1 a)), one fragment carrying the expression cassette for L (see Example 1 a)) and one fragment carrying the expression cassette for HAI-1 with a fourth fragment carrying the vector backbone elements in a four-way ligation reaction. In this way two different back vectors were generated either expressing HAI-1 under the control of the CMV promoter or the SV40 enhancer and early promoter. The expression cassette configuration (including the Cre-recombinase sites) of both final back vectors was LoxFas-H-L-HAI-1-2L.

Beside the expression cassettes of H, L and HAI-1 the back vectors contained
a sequence encoding puromycin acetyltransferase (SEQ ID NO: 02) lacking the start codon,
an origin of replication from the vector pUC18 which allows replication of this plasmid in E. coli, and
a beta-lactamase gene which confers ampicillin resistance in E. coli.

### c) BPTI Expression Constructs

In order to evaluate the effect of the protease inhibitor BPTI (aprotinin) on the expression of the bispecific antibody described in Example 1 a), vectors for the additional expression of BPTI (aprotinin) from a separate expression cassette were generated. The expression cassette configuration (including the Cre-recombinase sites) after targeted stable integration into the host genome was: L3-K-K-L-L-LoxFas-K-L-H-BPTI-2L.

### Expression Cassettes

The same immunoglobulin expression cassette configurations were used as described in Example 1 a).

For the expression of BPTI (aprotinin) a transcription unit comprising the following functional elements was used:
the enhancer and early promoter of simian virus 40 (SV40) (SEQ ID NO: 102),
signal peptide (SEQ ID NO: 175) coding sequence (SEQ ID NO: 176),
a nucleic acid encoding BPTI as pro-peptide (SEQ ID NO:177),
the bovine growth hormone polyadenylation sequence (BGH pA) (SEQ ID NO: 100), and
the human gastrin terminator (hGT) (SEQ ID NO: 101).

Beside the expression cassette the vector contained
an origin of replication from the vector pUC18 which allows replication of this plasmid in E. coli, and
a beta-lactamase gene which confers ampicillin resistance in E. coli.

### Front Vector

The front vector with the expression cassette configuration (including the Cre-recombinase sites) L3-K-K-L-L-LoxFas described in Example 1 a) was used.

### Back Vector

A DNA fragment coding for BPTI (aprotinin) was chemically synthesized and introduced into a shuttle plasmid using suitable restriction sites, thereby yielding the complete expression cassettes for BPTI (aprotinin). The expression cassette for BPTI (aprotinin) was excised in a way from the shuttle plasmid that it was provided with suitable sticky ends.

The back vector was generated by combining one DNA fragment carrying the expression cassette for H (see Example 1 a)), one fragment carrying the expression cassette for L (see Example 1 a)) and one fragment carrying the expression cassette for BPTI (aprotinin) with a fourth fragment carrying the vector backbone elements in a four-way ligation reaction. The expression cassette configuration (including the Cre-recombinase sites) of the final back vector was LoxFas-K-L-H-BPTI-2L.

Beside the expression cassettes of H, L and BPTI the back vector contained
a sequence encoding puromycin acetyltransferase (SEQ ID NO: 02) lacking the start codon,
an origin of replication from the vector pUC18 which allows replication of this plasmid in E. coli, and
a beta-lactamase gene which confers ampicillin resistance in E. coli.

### d) Puromycin Acetyltransferase-T2A-BPTI Fusion Expression Constructs

In order to evaluate the effect of the protease inhibitor BPTI (aprotinin) on the expression of the bispecific antibody described in Example 1 a), vectors for the additional expression of the BPTI amino acid sequence fused to the C-terminus of the puromycin-N-acetyltransferase amino acid sequence by means of a self-cleavable T2A peptide linker were generated. The expression cassette configuration (including the Cre-recombinase sites) after targeted stable integration into the host genome was: L3-K-K-L-L-LoxFas-K-L-H-puroT2A-BPTI-2L.

### Expression Cassettes

The same immunoglobulin expression cassette configurations were used as described in Example 1 a).

### Front Vector

The front vector with the expression cassette configuration (including the Cre-recombinase sites) L3-K-K-K-L-L-LoxFas as described in Example 1 a) was used.

### Back Vector

The same procedure as described in Example 1 a) was applied differing only in the back vector that was used. Here the back vector codes for a puromycin acetyltransferase-T2A-BPTI fusion protein instead of a puromycin acetyltransferase alone. Consequently, the expression cassette configuration (including the Cre-recombinase sites) of the final back vector was LoxFas-K-L-H-puroT2A-BPTI-2L.

Beside the expression cassettes of H and L the back vector contained
a sequence encoding a puromycin acetyltransferase-GSG-T2A-GGGGS-BPTI fusion protein (SEQ ID NO: 01-SEQ ID NO: 14-SEQ ID NO: 99-SEQ ID NO: 177) lacking the start codon (SEQ ID NO: 02-SEQ ID NO: 05-SEQ ID NO: 15-SEQ ID NO: 174-SEQ ID NO: 178),
an origin of replication from the vector pUC18 which allows replication of this plasmid in E. coli, and
a beta-lactamase gene which confers ampicillin resistance in E. coli.

### Example 2

### Cultivation of Host Cells:

### a) ExpiCHO-S

ExpiCHO-S cells (Thermo Fisher) were cultivated as recommended by the manufacturer.

### b) Expi293F

Expi293F cells (Thermo Fisher) were cultivated as recommended by the manufacturer.

c) CHO-K1 TI host cells

CHO-K1 TI host cells were cultivated at 37° C. in a humidified incubator with 85% humidity and 5% CO₂ in a proprietary DMEM/F12-based medium containing 300 μg/ml Hygromycin B and 4 μg/ml of a second selection marker. The cells were split every 3 or 4 days at a concentration of 0.3×10\hat{ }6 cells/ml in a total volume of 30 ml. For the cultivation 125 ml non-baffled Erlenmeyer shake flasks were used. Cells were shaken. The viable cell density was determined with a Cedex HiRes Cell Counter (Roche).

### Example 3

### Transfection:

### a) Transient Transfection of ExpiCHO-S and CHO-K1 TI Host Cells

For transient transfection and production, expression vectors coding for single immunoglobulin subunits were used at a molar ratio of 4 (K):1 (H):2 (L). Transfection of ExpiCHO-S was performed according to the manufacturer's protocol of the ExpiFectamine™ CHO Transfection Kit, prototype (A29128). CHO-K1 TI host cells were transfected using the MaxCyte STX electroporation device (MaxCyte Inc., Gaithersburg) with OC-400 electroporation cassettes according to the manufacturer's protocol. 3×10∂cells were transfected with a total of 30 μg nucleic acids. After transfection, the cells were seeded in 30 ml medium.

### b) Stable Transfection of CHO-K1 TI Host Cells by Targeted Integration

For stable transfection, equimolar amounts of front and back vectors were mixed. Total DNA used per transfection was 30 μg with plasmid ratio 2.5:2.5:1 (front-, back-, Cre-vector).

Two days prior to transfection CHO-K1 TI host cells were seeded in fresh medium with a density of 4×10≡cells/ml. Transfection was performed with the MaxCyte STX electroporation device (MaxCyte Inc., Gaithersburg) using OC-400 electroporation cassettes according to the manufacturer's protocol. 3×10∂cells were transfected with a total of 30 μg nucleic acid, i.e. either with 30 μg plasmid or with 5 μg Cre mRNA and 25 μg front- and back-vector mixture. After transfection, the cells were seeded in 30 ml medium without selection agents.

On day 5 after seeding the cells were centrifuged and transferred to 80 mL chemically defined medium containing puromycin (selection agent 1) and 1-(2′-deoxy-2′-fluoro-1-beta-D-arabinofuranosyl-5-iodo)uracil (FIAU; selection agent 2) at effective concentrations at 6×10≡cells/ml for selection of stable recombinant cells. The cells were incubated at 37° C., 150 rpm. 5% CO2, and 85% humidity from this day on without splitting. Cell density and viability of the culture was monitored regularly. When the viability of the culture started to increase again, the concentrations of selection agents 1 and 2 were reduced to about half the amount used before. Therefore, 4×10\hat{ }5 cells/ml were centrifuged and resuspended in 40 ml selection media II (chemically-defined medium, 12 selection marker 1 & 2). The cells were incubated with the same conditions as before and also not splitted.

Fourteen to twenty-one days after the selection had been started, the viability exceeded 90% and selection was considered as complete.

### Example 4

### Production of IgG-Like Proteins

### a) Transient Production of IgG-Like Proteins in ExpiCHO-S or CHO-K1 TI Host Cells

Cell supernatants of transiently transfected cells were harvested after 7 days by centrifugation and subsequent filtration (0.2 m filter), and proteins were purified from the harvested supernatant by standard methods as indicated below.
b) Transient Production of IgG-Like Proteins in Expi293F Cells

Cells were seeded in Expi293™ medium (Gibco, Cat. N^{o} 1435101) at a density of 2.5×10\hat{ }6/ml. Expression vectors and ExpiFectamine (Gibco, ExpiFectamine™ transfection kit, Cat. N^{o} 13385544) were separately mixed in OptiMEM™ reduced serum medium (Gibco, Cat. N^{o} 11520386). After some minutes both solutions were combined, mixed by pipetting and incubated further at room temperature. Cells were added to the expression vector/ExpiFectamine solution and incubated for 24 hours at 37° C. in a shaking incubator with a 5% CO2 atmosphere. One day post transfection, supplements (Transfection Enhancers 1 and 2, ExpiFectamine™ transfection kit) were added. Cell supernatants were harvested after 4-5 days by centrifugation and subsequent filtration (0.2 μm filter), and proteins were purified from the harvested supernatant by standard methods as indicated below.

### c) Stable Production of IgG-Like Proteins in CHO-K1 Cells

### Fed-Batch Cultivation

Fed-batch production cultures were performed in shake flasks with proprietary chemically defined medium. Cells were seeded at 2×10\hat{ }6 cells/ml. Cultures received proprietary feed medium on days 1, 3, and 6. Viable cell count (VCC) and percent viability of cells in culture was measured on days 0, 3, 7, 10, 12 and 14 using a Cedex HiRes (Roche Diagnostics GmbH, Mannheim, Germany). Product titer was measured on days 3, 5, 7, 10, 12 and 14 using a Cobas analyzer (Roche Diagnostics GmbH, Mannheim, Germany). The supernatant was harvested 10, 12 or 14 days after start of fed-batch by centrifugation (10 min., 1000 rpm followed by 10 min., 4000 rpm) and cleared by filtration (0.22 μm). Harvest titers were determined using protein A affinity chromatography with UV detection. Product quality was determined by Caliper's LabChip (Caliper Life Sciences).

### High Cell Density Fed-Batch Cultivation

High cell density fed-batch production cultures were performed in Ambr 250 vessels (Sartorius Stedim) with proprietary chemically defined medium. Cells were seeded at 15×10\hat{ }6 cells/ml on day 0. Cultures received proprietary feed medium on days 1, 3 and 6. Viable cell count (VCC) and percent viability of cells in culture was measured on days 0, 3, 7, 10, 12 and 14 using a Cedex HiRes instrument (Roche Diagnostics GmbH, Mannheim, Germany). Product titer was measured on days 3, 5, 7, 10, 12, and 14 using a Cobas Analyzer (Roche Diagnostics GmbH, Mannheim, Germany). The supernatant was harvested 10 or 12 or 14 days after start of the cultivation by centrifugation (10 min., 1000 rpm followed by 10 min., 4000 rpm) and cleared by filtration (0.22 μm). Harvest titers were determined using protein A affinity chromatography with UV detection. Product quality was determined by Caliper's LabChip (Caliper Life Sciences).

### Example 5

### Protein Purification:

Recombinant immunoglobulin-like proteins were purified from cell culture supernatants by affinity chromatography using MabSelectSure-Sepharose™ (GE Healthcare, Sweden) chromatography. Sterile filtered cell culture supernatants were captured on a MabSelect SuRe resin equilibrated with PBS buffer (10 mM sodium phosphate, 1 mM potassium phosphate, 137 mM sodium chloride and 2.7 mM potassium chloride, pH 7.4), washed with equilibration buffer, eluted with 25 mM citrate buffer, pH 3.0 and neutralized with 1 M Tris pH 9.

### Example 6

### Analytical Methods

### a) Titer Determination

The concentration of recombinant immunoglobulins in cell culture supernatants was quantitatively measured by affinity HPLC chromatography. Briefly, centrifuged and sterile filtered cell culture supernatants were applied to a Poros A/20 column (Applied Biosystems) in 200 mM KH₂PO₄, pH 7.4 and eluted with 200 mM NaCl, 100 mM citric acid, pH 2.5 on a Dionex Ultimate HPLC system (Thermo Fisher Scientific). The eluted antibody was quantified by UV absorbance and integration of peak areas. A purified standard IgG1 antibody served as a standard.

### b) CE-SDS

Product purity and integrity were analyzed by CE-SDS using microfluidic Labchip technology (PerkinElmer, USA) under reducing and non-reducing conditions. For this purpose, 5 μl of sample solution was prepared using the HT Protein Express Reagent Kit according to the manufacturer's instructions and analyzed on LabChip GXII system using a HT Protein Express Chip. Data were analyzed using LabChip GX Software.

### c) SEC

Size exclusion chromatography (SEC) for the determination of the aggregation and oligomeric state of recombinant immunoglobulins was performed by HPLC chromatography. Briefly, protein A purified product was applied to a TSKgel QC-PAK GFC 300 column (Tosoh Bioscience) or to a Tosoh TSKgel UP-SW3000 column in 250 mM KCl, 200 mM K₂HPO₄/KH₂PO₄ buffer (pH 6.2) on a Dionex Ultimate® HPLC system (Thermo Fisher Scientific). The eluted antibody was quantified by UV absorbance and integration of peak areas. BioRad Gel Filtration Standard #151-1901 served as a gel filtration calibration standard

1. A nucleic acid comprising in operably linked form, elements
a) a nucleic acid encoding a selection marker,
b) a nucleic acid encoding a self-cleaving peptide sequence, and
c) a nucleic acid encoding a proteinaceous protease inhibitor.

2. The nucleic acid according to claim 1, wherein the elements in 5′- to 3′-direction have the sequence of a)-b)-c).

3. The nucleic acid according to claim 1, wherein the nucleic acid further comprises in operably linked form the following elements
d) a promoter upstream (5′) to the first element,
e) a polyadenylation signal sequence downstream (3′) to the last element,
f) optionally a terminator sequence downstream (3′) to the nucleic acid of e).

4. The nucleic acid according to claim 1, wherein the nucleic acid encoding a selection marker encodes puromycin acetyltransferase or a functional variant thereof capable of inactivating/modifying puromycin.

5. The nucleic acid according to claim 4, wherein
the nucleic acid encoding a selection marker has the nucleotide sequence of SEQ ID NO: 02, or
the nucleic acid encoding a selection marker is a variant of the nucleotide sequence of SEQ ID NO: 02 encoding a selection marker that has the amino acid sequence of SEQ ID NO: 01, or
the nucleic acid encoding a selection marker encodes a functional variant of SEQ ID NO: 01 capable of inactivating/modifying puromycin.

6. The nucleic acid according to claim 1, wherein the self-cleaving peptide sequence is T2A or a functional variant thereof capable of effecting ribosome skipping.

7. The nucleic acid according to claim 6, wherein
the nucleic acid encoding a self-cleaving peptide sequence has the nucleotide sequence of SEQ ID NO: 15, or
the nucleic acid encoding a self-cleaving peptide sequence is a variant of the nucleotide sequence of SEQ ID NO: 15 encoding a self-cleaving peptide sequence that has the amino acid sequence of SEQ ID NO: 14, or
the nucleic acid encoding a self-cleaving peptide sequence encodes a functional variant of SEQ ID NO: 14 capable of effecting ribosome skipping.

8. The nucleic acid according to claim 1, wherein the nucleic acid encoding a proteinaceous protease inhibitor encodes BPTI or a functional variant thereof capable of inhibiting one or more serine proteases.

9. The nucleic acid according to claim 8, wherein
the nucleic acid encoding a proteinaceous protease inhibitor has the nucleotide sequence of SEQ ID NO: 87 or SEQ ID NO: 178, or
the nucleic acid encoding a proteinaceous protease inhibitor is a variant of the nucleotide sequence of SEQ ID NO: 87 encoding a proteinaceous protease inhibitor that has the amino acid sequence of SEQ ID NO: 86, or
the nucleic acid encoding a proteinaceous protease inhibitor encodes a functional variant of SEQ ID NO: 86 capable of inhibiting one or more serine proteases.

10. The nucleic acid according to claim 3, wherein the promoter is the SV40 promoter.

11. A cell comprising the nucleic acid according to claim 1.

12. The cell according to claim 11, wherein the cell further comprises one or more nucleic acid sequences encoding a heterologous polypeptide.

13. The cell according to claim 12, wherein the heterologous polypeptide is an antibody that comprises one or more protease-cleavable amino acid sequences.

14. The cell according to claim 13, wherein the cell is a CHO-K1 cell.

15. A method for producing a heterologous polypeptide in a recombinant cell, wherein the method comprises the following steps:
cultivating a cell according to claim 12 in a cultivation medium to produce the heterologous polypeptide,
recovering the heterologous polypeptide from the cell or the cultivation medium, and
optionally purifying the heterologous polypeptide by one or more chromatography steps.

16. The method according to claim 15, wherein the amount of recovered not-cleaved heterologous polypeptide is increased compared to a method wherein a cell not comprising a nucleic acid according to claim 1 is used.

17. Use of a nucleic acid according to claim 1 for reducing protease cleavage of a heterologous polypeptide during recombinant production in a mammalian cell.
